(19) United States
(12) Patent Application Publication
(10) Pub. No.: US 2010/0190201 A1
(43) Pub. Date: Jul. 29, 2010

(54) Methods

(75) Inventors: Philip Howlett Jones, Cambridge (GB); Benjamin David Simons, Cambridge (GB); Allon Klein, Cambridge (GB)

Correspondence Address: MCANDREWS HELD & MALLOY, LTD, 500 WEST MADISON STREET, SUITE 3400, CHICAGO, IL, 60661, US

(73) Assignees: MEDICAL RESEARCH COUNCIL, London (GB); CAMBRIDGE ENTERPRISE LIMITED, Cambridge (GB)

(21) Appl. No.: 12/668,148

(22) PCT Filed: Jul. 11, 2008
(86) PCT No.: PCT/GB08/02387

§ 371 Date: Jan. 7, 2010

(30) Foreign Application Priority Data
Jul. 13, 2007 (GB) 0713673.2

(51) Int. Cl. C12Q 1/02

(52) U.S. Cl. 435/29

(57) ABSTRACT

The invention relates to a method of detecting an altered behaviour in a population of cells, said method comprising determining at least one of the following characteristics of the population of cells; (i) the proportion of stem cells, proliferating cells and differentiated cells in said cell population; or (ii) the size of stem cell clusters in said cell population; or (iii) the separation of stem cell clusters in said cell population; and comparing said at least one characteristic to a reference value, wherein a difference between the determined value and the reference value indicates an altered behaviour in said population of cells. Preferably the cells are mammalian, more preferably human epithelial cells, more preferably human epidermal cells.

### FIELD OF THE INVENTION

The invention is in the field of analysis of cell behaviour. In particular the invention relates to methods of analysing and modelling cell behaviour in homeostatic systems, and to techniques for detecting and studying perturbation or manipulation of such behaviour. More in particular the invention relates to application of such methods to mammalian epithelial cells such as human epidermal cells.

### BACKGROUND TO THE INVENTION

Mammalian epidermis is organized into hair follicles interspersed with interfollicular epidermis (IFE), which consists of layers of keratinocytes (Fuchs, 2007). In IFE, proliferating epidermal progenitor cells are found in the basal cell layer. On commitment to terminal differentiation, basal cells exit the cell cycle and subsequently migrate into the suprabasal cell layers. Stem cells, which may be defined as cells that retain their ability to proliferate and generate progeny that will ultimately undergo terminal differentiation, are found hair-follicle bulge, but these cells appear to play no part in maintaining normal IFE (Ito et al., 2005; Levy et al., 2005; Morris et al., 2004; Tumbar et al., 2004). There is also evidence for the existence of stem cells in both mouse and human IFE, although these are largely quiescent (Barrandon and Green, 1987; Bickenbach, 1981; Bickenbach and Chism, 1998).

It has long been held that mammalian epidermis is maintained by two populations of cells (Potten, 1981). Long lived slowly cycling stem cells have been proposed to generate a short lived population of transit amplifying (TA) cells which differentiate after a limited number of cell divisions. Despite its wide application in the literature, the evidence in support of this stem/TA cell model is indirect and ambiguous, which is a problem in the art.

Recently, access to clonal fate data obtained through inducible genetic labelling has revealed an alternative mechanism of epidermal homeostasis in the tail skin of adult mice (Clayton et al., 2007). These results demonstrate that normal adult IFE is maintained by a single population of committed progenitor or CP cells, committed to terminal differentiation but able to undergo an unlimited number of cell divisions generating equal numbers of cycling or post mitotic daughters by symmetric and asymmetric cell divisions. Although these findings shed light on the mechanism of epidermal maintenance, they leave open the question of the function of stem cells in IFE. In addition, the discovery of a new paradigm of stem-cell independent tissue maintenance in mouse raises the question as to whether similar rules may govern the behaviour of human keratinocytes. Thus, understanding of stem cell function in mammalian epidermis remains limited in the art, which is a problem.

Whilst in the mouse stem cells are scattered throughout the IFE, in humans stem cells lie in cohesive clusters, interspersed by cycling and differentiating cells with a much lower proliferative potential (Braun et al., 2003; Jensen et al., 1999; Jones et al., 1995). Stem cells may be identified by their rapid adhesion to extracellular matrix proteins and their high level of expression of the β1 integrin family of extracellular matrix receptors and other markers such as the cell surface proteoglycan MPSG and the transcription factor LRIG1 (Jensen and Watt, 2006; Jones and Corwin, 1993; Jones et al., 1995; Jones and Watt, 1993; Legg et al., 2003). Immunostaining of human epidermal whole-mounts with the proliferation markers Ki67 and BrdU reveals that cells in the β1 integrin-high clusters appear almost completely quiescent (Jensen et al., 1999; Jones et al., 1995). However, when cultured at clonal density, single human epidermal stem cells generate large growing colonies, which exhibit a very high proliferative potential when subcloned (Barrandon and Green, 1987). By contrast, proliferating and terminally differentiated basal cells are found lying between stem cell clusters (Jensen et al., 1999). These cells are slowly adherent when plated onto extracellular matrix proteins, express low levels of β1 integrin MPSG and LRIG and only generate small clones in culture, all of the cells in which undergo terminal differentiation (Jensen and Watt, 2006; Jensen et al., 1999; Jones et al., 1995; Legg et al., 2003).

Interpreted within the framework of the stem/TA hypothesis, the β1 integrin-low cells have been interpreted as TA cells. However, looking beyond this superficial identification, several issues challenge the viability of the stem/TA cell hypothesis. Firstly, the limited renewal capacity of TA cells requires the stem cell population to remain in cycle. It is, therefore, striking that the vast majority of cells in the integrin-bright clusters appear to be non-cycling, yet are readily recruited into cycle when transferred to culture (Jensen et al., 1999; Jones et al., 1995). Secondly, without further regulation, it seems impossible to explain the existence and stability of stem cell patterning within the framework of the stem/TA cell hypothesis (Jones et al., 1995). Thus, there are serious drawbacks with the current understanding of maintenance of mammalian epidermis. Furthermore, the degree to which stem cells are involved or participate in this maintenance is far from clear.

Animal testing, such as animal testing of cosmetics, is increasingly unpopular. Indeed, recent changes to European law mean that an effective ban on animal testing of cosmetics across the European Union will enter into force during 2008. Thus, there is a need in the art for systems for the assessment of the effect of compounds or treatments on animal cells without the need for testing on animals.

The present invention seeks to overcome problem(s) associated with the prior art.

### SUMMARY OF THE INVENTION

The understanding of murine epidermal homeostasis has recently been significantly overhauled (Clayton et al 2007). In this revised model, it is clear that committed progenitor, cells (CPCs) are key in the maintenance of tissue homeostasis. Indeed, a striking feature of that model is that stem cell compartments, if indeed they even exist in some tissue compartments such as murine interfollicular epidermis, play no detectable role in tissue homeostasis. By contrast, the present inventors have studied the interplay between stem cells, actively proliferating cells, and post mitotic terminally differentiated cells.

The present inventors have now focused on human epidermis as a model epithelial system. Human epidermis in homeostasis includes a population of stem cells. In contrast to the mouse system, human epidermal stem cells are arranged in particular patterns or clusters. The stem cells within these clusters are quiescent. Visualising these quiescent stem cells in the basal layer of the epidermis reveals a “chessboard like” pattern of clusters of dormant stem cells surrounded by a sea of proliferating or terminally differentiated (post mitotic) basal cells. Indeed, it is remarkable that in vitro, a single stem cell can go on to divide and to make a cohesive patch of epidermal cells with stem cell patterning closely resembling that observed in vivo.

Through the study of this complicated epidermal system, the inventors have developed a mathematical model describing the patterning behaviour. Despite the considerable complexity of the biological system under study, the descriptive model can be expressed in relatively straightforward mathematical terms. Indeed, it is surprising to note this model is based on hydrodynamics, principles of surface tension and the differing propensities of different cell types to interact with one another physically. Among other things, this model reveals the striking insight that the organisational or patterning behaviour of the cells can be seen as spinodal decomposition.

The invention is based on these surprising findings.

Thus, in one aspect, the invention relates to a method of detecting an altered behaviour in a population of cells, said method comprising determining at least one of the following characteristics of the population of cells;
(i) the proportion of stem cells, proliferating cells and differentiated cells in said cell population; or
(ii) the size of stem cell clusters in said cell population; or
(iii) the separation of stem cell clusters in said cell population; and comparing said at least one characteristic to a reference value,

wherein a difference between the determined value and the reference value indicates an altered behaviour in said population of cells.

Suitably two or more of said characteristics are determined, more suitably all three said characteristics are determined. Suitably determination is by direct or indirect measurement. Clearly, the three characteristics are related by the model of the invention. Thus, given any two, the third can be inferred. This is an advantage of measuring at least two of the three characteristics. Thus suitably at least two characteristics are measured.

More suitably the third characteristic is not inferred but is rather measured, which advantageously validates the accurate measurement of the other two characteristics and leads to a more robust analysis. For example, the inferred value of the third characteristic can be compared to the measured value of the third characteristic—a finding that they are in agreement thereby validates the measurements. Thus suitably all three characteristics are measured.

The reference value is a value determined for a control population of cells. Altered behaviour is relative to the reference value i.e. relative to the control cells. Thus in one sense altered behaviour simply means different from the reference value/control population. Suitably these cells are normal cells of the same type as those being analysed for altered behaviour. In this way, altered behaviour should advantageously be clearly detected. The reference value(s) may be determined in parallel e.g. simultaneously with determination of the value(s) for the population of interest, or may be a previously determined reference value for a given cell type.

Suitably said behaviour is selected from the group consisting of stem cell division rate, stem cell differentiation rate, stem cell adhesion capacity, committed progenitor cell division rate and differentiated cell migration rate.

Suitably when the characteristic determined is the size of stem cell clusters in the population, it is determined as the average diameter of stem cell clusters in millimetres or microns (μm), preferably microns.

Suitably when the characteristic determined is the separation of stem cell clusters in the population, it is determined as the average distance between the outer edges of discrete adjacent stem cell clusters in millimetres.

Suitably said population of cells is a population of mammalian epidermal cells. Suitably said population of cells is a population of basal layer cells. Suitably said population of cells comprises an organotypic keratinocyte culture. Suitably said population of cells comprises a submerged culture (eg. as in Jones et al 1995).

Suitably said cells are human cells.

Suitably the cells are primary cells.

In another aspect, the invention relates to a method as described above wherein said reference value is generated from a control population of cells.

In another aspect, the invention relates to a method for assessing the effect of a treatment on behaviour in a population of cells, said method comprising
(i) providing a first and a second population of cells;
(ii) applying the treatment to said first population of cells;
(iii) incubating said first and second populations of cells;
(iv) detecting an altered behaviour in said first population of cells as described above,
wherein said reference value is the value determined for said second population of cells, and wherein detection of altered behaviour in said first population of cells indicates that said treatment has an effect on behaviour in said population of cells. Incubation is purely to allow the treatment to have the effect. Typically incubation will be determined to provide sufficient time for the pattern to form or to change or for the characteristic being determined to have changed or become stable depending on the application or the characteristic being determined. Incubation times may be long enough to permit continued cell division/migration as desired by the operator.

Treatment may be genetic, environmental, infectious, chemical, physical eg. temperature or any other kind of treatment whose effect it is desired to assess. Typically the treatment will be application of a chemical or pharmaceutical agent (eg. candidate drug) to the culture medium in which the cells are being maintained. Clearly this may need to be reapplied or ‘topped up’ depending on the incubation time, the stability in the medium, whether it is a transient or long-term treatment or other such considerations well within the abilities of the skilled operator.

In another aspect, the invention relates to a method as described above wherein said reference value is predicted or described by the equation

$$\frac{d {c}_{X}}{d t} = - ∇ \cdot {J}_{X} + {R}_{X} , \\ where {J}_{X} = - {\sum }_{Y = S , A , B , Φ}^{ } {M}_{XY} ∇ ( \frac{δ F}{{δc}_{Y}} ) . \qquad ( 1 )$$

### DETAILED DESCRIPTION OF THE INVENTION

Protecting adult tissue stem cells from genetic damage is essential to diminish the risks of stem cell senescence and malignant transformation. Patterning protects human epidermal stem cells. Recently inducible genetic labelling has revealed that interfollicular epidermis in mice is maintained by a single population of progenitor cells enabling stem cells to remain quiescent in normal homeostasis (Clayton et al., 2007). Here we investigate human interfollicular epidermis, which is organised into clusters of largely quiescent cells stem cells separated by proliferating and differentiating keratinocytes (Jensen et al., 1999; Jones et al., 1995). Remarkably the stem cell clusters are reproduced in culture, where the clonal progeny of a single stem cell recreate the distribution of stem cells seen in vivo (Jones et al., 1995). With reference to the properties of the murine system, we develop a hydrodynamic theory of epidermal maintenance, particularly human epidermal maintenance. This hydrodynamic theory explains stem cell fate, and the origin, dynamics and stability of the observed patterning. To test and demonstrate the utility of the predictions made by the model, we have further explored the real-time dynamics of cultured sheets of keratinocytes. As well as challenging the accepted function of the transit-amplifying cell compartment put forward in the art, these results identify a natural mechanism of self-regulation that isolates and protects the stem cells by allowing them to remain quiescent in normal homeostasis whilst remaining accessible as a resource for tissue repair.

According to the present invention there is presented a model of cell behaviour. In particular, the model is based on ideas about the adhesiveness of stem cells both to the matrix and to each other. With a surprisingly minimal set of assumptions such as that once there are ‘enough’ cells that they stop dividing, then a robust model of cell behaviour can be developed to describe such systems. An example of the predictions made by this model is presented in FIG. 2C—discussed in more detail in the examples section. A key tenet is that the hydrodynamic model presented herein gives rise to strong predictions regarding the effects of patterning. This permits the analysis and understanding of cell behaviours and in particular alterations or perturbations of such behaviour. These applications of the invention are useful in understanding and studying cell behaviour and are particularly useful in the study or assay of effects of particular compounds or treatments on cell behaviour, such as in toxicity testing, testing for carcinogenic, teratogenic or other adverse effects which can be read out by monitoring the effect(s) on cell behaviour according to the present invention.

### DEFINITIONS

A ‘cluster’ or ‘stem cell cluster’ as used herein refers to a group of cells which is enriched for stem cells. This term should not be interpreted to imply the total exclusion of non-stem-cells from the cluster, but rather should be understood to refer to a recognisable zone or area comprised predominantly of stem cells. Such areas may be detected or defined by visualising the stem cells within the cell population being examined. This visualisation may be by any suitable means known in the art. Examples of such means are described herein.

In this context, stem cells are cells which are not committed to a long-term differentiated fate, persist within a tissue long term and have the potential to undergo unlimited cell divisions which generate cells that are stem cells themselves.

Transit amplifying cells are cells which only divide a finite number of times. As used herein the term “transit amplifying cell” is consistent with the established Potten model. The number of cell divisions which a transit amplifying cell can undergo is finite and is fixed. There is no evidence to support the existence of transit amplifying cells as defined by Potten.

A committed progenitor cell is a cell committed to terminal differentiation which may undergo an unlimited number of cell divisions prior to all of its progeny exiting the cell cycle. In contrast to the transit amplifying cells, which are considered to have a “memory” involved in restricting the number of further cell divisions which they undergo, committed progenitor cells have no such restriction. There is no limit to the number of divisions which a committed progenitor cell may undergo. Each division has a chance of symmetry or non-symmetry as determined for that particular cellular context.

### Cell Markers

The cell types are described throughout the specification. In order to determine what particular cell types are present, the cells can be stained or visualised according to techniques known in the art. In particular, the following examples of markers indicative of different cell types are provided. Clearly any suitable marker of the particular cell type or particular indicative property (eg. whether or not cells are actively cycling) may be employed; the following table represents exemplary markers and/or properties which are useful in the present invention.

| Cell Type | Marker | Reference |
|---|---|---|
| Stem | One or more of | |
| | High β1 integrin | 1-3 |
| | High Delta 1 | 4 |
| | High MSPG | 5 |
| | High LRIG1 | 6 |
| | Low DSG3 | 7 |
| Cycling progenitor cell | One or more of | |
| (Committed Progenitor | Low β1 integrin | |
| Cell or CPC) (=‘A’ cell) | Low Delta 1 | |
| | Low MSPG | |
| | Low LRIG1 | |
| | High DSG3 | |
| | AND | |
| | Expression of a cell cycle | |
| | marker, eg | |
| | CDC6 | 8, 9 |
| | Ki67 | |
| Post mitotic basal cell | One or more of | |
| (terminally differentiated | Low β1 integrin | |
| cell) | Low Delta 1 | |
| | Low MSPG | |
| | Low LRIG1 | |
| | High DSG3 | |
| | AND | |
| | Negative for cell cycle | |
| | marker, eg CDC6 or Ki67 | |

1. Jones, P. H., Harper, S. & Watt, F. M. Stem cell patterning and fate in human epidermis. Cell 80, 83-93 (1995).
2. Jones, P. H. & Watt, F. M. Separation of human epidermal stem cells from transit amplifying cells on the basis of differences in integrin function and expression. Cell 73, 713-24 (1993).
3. Jensen, U. B., Lowell, S. & Watt, F. M. The spatial relationship between stem cells and their progeny in the basal layer of human epidermis: a new view based on whole-mount labelling and lineage analysis. Development 126, 2409-18 (1999).
4. Lowell, S., Jones, P., Le Roux, I., Dunne, J. & Watt, F. M. Stimulation of human epidermal differentiation by delta-notch signalling at the boundaries of stem-cell clusters. Curr Biol 10, 491-500 (2000).
5. Legg, J., Jensen, U. B., Broad, S., Leigh, I. & Watt, F. M. Role of melanoma chondroitin sulphate proteoglycan in patterning stem cells in human interfollicular epidermis. Development 130, 6049-63 (2003).
6. Jensen, K. B. & Watt, F. M. Single-cell expression profiling of human epidermal stem and transit-amplifying cells: Lrigl is a regulator of stem cell quiescence. Proc Natl Acad Sci USA 103, 11958-63 (2006).
7. Wan, H. et al. Desmosomal proteins, including desmoglein 3, serve as novel negative markers for epidermal stem cell-containing population of keratinocytes. J Cell Sci 116, 4239-48 (2003).
8. Madine, M. A. et al. The roles of the MCM, ORC, and Cdc6 proteins in determining the replication competence of chromatin in quiescent cells. J Struct Biol 129, 198-210 (2000).
9. Williams, G. H. et al. Improved cervical smear assessment using antibodies against proteins that regulate DNA replication. Proc Natl Acad Sci USA 95, 14932-7 (1998).

### Mammalian Epidermis

Mammalian epidermis is discussed herein as a general epithelial model. Mammalian epidermis is also a preferred system to which the invention is applied. There are 3 types of cell in mammalian epidermis:

Adult tissue stem cells. Traditionally regarded as cells that persist throughout life and self renew, dividing to generate stem cells and differentiating to form committed progenitor cells. These are contained within the ‘β1 integrin high’ cell clusters.

Committed progenitor (CP) cells. These are the population that maintains normal epidermal homeostasis, so that the stem cells (known to be located in clusters in interfollicular epidermis in human and in the hair follicles in mice) can remain out of cycle. Their behaviour described in Clayton et al (2007) is such that the CP population is self maintaining, but that all the progeny of a CP cell will ultimately terminally differentiate, so they do not fulfil the definition of stem cells. In mouse all or almost all the cycling cells in the IFE are CP cells. In human CP cells lie between the stem cell clusters, ie. are β1 integrin low.

Post mitotic basal cells. Have irreversibly exited the cell cycle, waiting to leave the basal layer. In mouse lie scattered amongst CP cells. In human these cells are scattered amidst the CP cells between the stem cell clusters. Are β1 integrin low and negative for cell cycle markers.

Alternatively, or in addition, the following guide may be used in particular for epidermis/keratinocytes:

| MCSP | TYPE | Ki67 | validate/control |
|---|---|---|---|
| MCSP positive | stem | | keratin 10 negative |
| MCSP negative | post mitotic | Ki67 negative | keratin 10 positive |
| MCSP negative | committed | Ki67 positive | keratin 10 negative |
| | progenitor | | |

These classifications may be used in determining the relative fraction of stem cells according to the present invention.

### Advanced Modelling of Tissue Homeostasis

In the following, we will show that the problem of IFE maintenance in mouse and human can be embraced within the framework of a single model providing a consistent explanation for the mechanism and stability of stem cell patterning, and the quiescence of the stem cell population in the normal adult system.

To develop a detailed “microscopic” theory of cell fate, one could try to draw on the many regulatory processes known to influence the function of cells in human epidermis (see, e.g., (Savill, 2003; Savill and Sherratt, 2003)). However, given the apparent complexity of the epidermal system, such a “first principles” approach is, in our view, at best overspecified and unreliable and, at worst, uncontrolled. In the following, we adopt a different approach placing emphasis on the constraints imposed by the experimental phenomenology. In particular, we propose that the large-scale structure and rigidity of the patterned cell distribution suggests that its origin is rooted in the collective properties of the system, which can be captured by a simpler hydrodynamic description. More specifically, we apply a particular microscopic model of cell fate as a means to engineer a generic and testable theory of mammalian tissue maintenance such as epidermal maintenance.

### Stem/CP Model

Focusing on the basal layer, we considered a cell population comprised of stem, committed progenitor, and post-mitotic cells. To account for the steric repulsion of cells, as evidenced by the near-uniform cell density of the basal layer, we will characterise the basal layer as a regular lattice of sites, each capable of hosting at most one of the three cell types. To regulate the cell density, we will allow progenitor cells (stem or CP) to divide only when neighbouring a site vacancy. Crucially, other regulatory mechanisms, such as through morphogen gradients or the mechanical stress-based control, translate to the same large-scale hydrodynamic behaviour. Motivated by studies of murine IFE maintenance, and the observed behaviour of human keratinocytes in culture, we will assume that CP cell division may lead to symmetric or asymmetric cell fate (FIG. 1e), while the migration of post-mitotic cells from the basal layer leads to the creation of vacancies (FIG. 1f) which are free to diffuse through the basal layer via the displacement of neighbouring cells (FIG. 1g). Turning to the stem cell compartment, as well as different regulatory pathways, one may conceive of several possible “channels” of division and differentiation. In the following, we will suppose that stem cells may undergo symmetric division or they may differentiate to form a progenitor cell committed to terminal differentiation (FIG. 1d). Once again, the generalisation to include further channels of symmetric or asymmetric division will lead to the same large-scale behaviour, the target of the present study. Finally, to facilitate the motion of cells in the basal layer, we will allow for diffusion processes which allow cells to exchange with their neighbours. Stem cells are more adherent to underlying extracellular matrix proteins than other basal cells by virtue of expressing the express high levels of functional β1 integrins (Jones and Watt, 1993). Stem cells also express high levels of the Notch receptor Delta (Lowell et al., 2000): Delta-notch signalling promotes stem-cell cohesiveness and inhibits stem cell motility (Lowell et al., 2000; Lowell and Watt, 2001). We therefore postulate that stem cell motion is constrained relative to other basal cells by the adhesiveness of stem cells to the underlying basement membrane and their neighbours (FIG. 1g).

Altogether, the processes summarised in FIG. 1d-g describe a complex composite cellular system. However, one may gain insight into the collective behaviour by isolating separate components of the dynamics:
Firstly, when the stem cell population is rendered quiescent (viz. γ_{SS}=γ_{SA}=0), the survival of the cell population demands that the symmetric division rates associated with the CP cell population are equal, γ_{AA}=γ_{BB}. In this case, one may show that the cell population conforms to the stem/CP model of murine IFE maintenance introduced/outlined above (eg. Clayton et al., 2007).
Secondly, a suppression of all channels of cell division and differentiation leads to a constrained “hard-core” diffusion of the basal layer cells. In this case, the adhesion properties of the stem cell compartment lead to a gradual segregation of cells through the development of dense, stem cell-rich, clusters of ever-increasing size through the process of “spinodal decomposition”. In the absence of division processes, this segregation would proceed unchecked until phase separation between a stem cell-rich domain and the remaining cells and vacancies was complete (Savill and Sherratt, 2003). Crucially, the inclusion of cell division and differentiation processes has the effect of “arresting” cluster growth.

Thus, according to one aspect of the present invention, formation of stable stem cell clusters may be understood as reflecting the balance between the depletion of stem cells within a cluster through their differentiation into committed progenitor cells, and their self-renewal through symmetric division concentrated along the more “compressible” (i.e. vacancy-rich) cluster edges. Once excluded from the dense stem cell cluster, the newly-differentiated stem cells add to the CP cell population thereby maintaining the surrounding regions of the basal layer and, through the pathway of terminal differentiation and migration, the supra-basal layers. The combined effect of the exclusion of CP cells from cohesive stem cell-rich clusters, and the migration of post-mitotic cells out of the stem cell-depleted regions results in an effective repulsion between neighbouring clusters leading to large-scale (irregular) pattern; formation reminiscent of the pattern structures observed in experiment (Jensen et al., 1999; Jones et al., 1995).

### Cluster Size

One of the key characteristics of cell populations which can be measured is the cluster size. The cluster size suitably refers to the size of the clusters of stem cells. The size may be expressed or measured in any suitable unit. For example, the cluster volume may be measured. The area of the cluster may be measured, in three dimensions or more suitably in two dimensions. The number of cells in the cluster (ie. number of cells per cluster) may be determined. The diameter of the cluster may be determined. When measuring linear characteristics of the cluster such as the diameter, this may be accomplished in absolute terms (for example in millimetres or microns (μm)) or may be accomplished in other suitable units such as the number of cell widths. Thus, the skilled operator will typically determine the mode by which cluster size is estimated or determined. To the extent necessary, units should clearly be converted to those appropriate for use with the mathematical model presented herein.

It is a key principle of the present invention that the hydrodynamic model presented herein remains true from whatever perspective a system is studied. As will be clear to the skilled reader, some of the modelling presented herein involves an assumption that a progenitor cell will divide only when a neighbouring site is vacant. However, other regulatory mechanisms such as the inference of morphogen gradients or indeed mechanical stress based control of proliferation are equally compatible with the same large-scale hydrodynamic behaviour discussed herein. Indeed, it is a striking revelation disclosed herein that systems assumed to be controlled by these alternative regulatory mechanisms still translate to the same large-scale hydrodynamic behaviour described herein. This is a key advantage of the invention, namely that the cell behaviours and predictions which can now be made based on the methods and techniques taught herein are in fact based on a fundamental description of the system of human epidermal homeostasis, and therefore have broad applicability within that setting and in other applications as will be apparent in light of the guidance provided.

### Hydrodynamics

Although, referring to the results of numerical simulation, one may see that the stem/CP cell model provides a seemingly sound microscopic basis to explain the organisation and activity of cells in IFE, it does not provide insight into the underlying mechanism of pattern formation nor its rigidity. We may turn to a long-range “hydrodynamic” description of the system which, in surrendering information about local fluctuations (i.e. at the level of the individual cells), addresses the collective behaviour of the coarse-grained cell densities (see ‘Hydrodynamics Model’ section). This advantageously enables questions about sensitivity of the behaviour to the rules of stem cell fate, about what can be gleaned from the morphology of the clusters (their typical size and separation), and about predictions regarding function and repair when the tissue is damaged or driven far away from steady-state (as in long term or immortalised culture) to be addressed. This is demonstrated in the examples section.

Clearly, the coincidence of experiment and theory in the steady-state system is reassuring, and a positive demonstration of the value and application of the system. Moreover, the advantageous strength and viability of the model hinges on its predictive power. As well as the steady-state characteristics, the model makes strong predictions about the dynamics of the system when displaced from steady-state either through natural means, such as injury or, artificially, as with experiments carried out in culture. Displaced from steady-state, a stability analysis of the hydrodynamics shows activation of the stem cell compartment until tissue homeostasis is restored.

Thus, we disclose a robust mechanism of pattern formation in human interfollicular epidermis that addresses much of the experimental phenomenology and provides a direct link to the observed properties of other mammalian systems such as the murine system. In adult, IFE is maintained largely by a committed progenitor cell population, allowing the majority of the stem cell population to remain quiescent. When driven away from steady-state through injury, the stem cell population can mobilise rapidly to replenish the CP cell population and, with it, the remaining tissue. As well as providing an explanation for the spatial organisation and quiescence of stem cell-rich clusters in the steady-state system, the model also captures the dynamics of the in vitro system, including the short-time scale aggregation of stem cells (eg. determined as β1 integrin positive cells) and, crucially, the pattern reconstruction observed in clonal colonies. As well as providing a natural explanation for the stability of the patterned distribution, and the attendant implications for tissue repair, it is interesting to note that the self-regulation implied by the model provides a mechanism for protection of the stem cell niche against aging, loss, and mutation.

### Hydrodynamics Model

Here we set out the hydrodynamics of the basal layer cell densities. Taken together, the stem/CP cell model describes a complex, multi-component, birth-death-diffusion process. However, for the coarse-grained system, the long-ranged properties can be captured by a hydrodynamics of Cahn-Hilliard type (A. J. Bray, Advances in Physics, 51, 481 (2002)). Defining c_{S}(r,t), c_{A}(r,t), and c_{B}(r,t) as, respectively, the local density of stem, CP, and post-mitotic cells, with c_{Φ}(r,t)=1−c_{S}(r,t)−c_{A}(r,t)−c_{B}(r,t) the vacancy density, the corresponding coupled set of reaction-diffusion equations take the form,

$$\frac{d {c}_{X}}{d t} = - ∇ \cdot {J}_{X} + {R}_{X} , \\ where {J}_{X} = - {\sum }_{Y = S , A , B , Φ}^{ } {M}_{XY} ∇ ( \frac{δ F}{{δc}_{Y}} ) . \qquad ( 1 )$$

Here, M_{XY}=σc_{X}(δ_{XY}−c_{Y}) denotes the mobility tensor, while the hard-core diffusion follows the effective chemical potential gradient,

$$∇ ( \frac{δ F}{{δc}_{Y}} ) ,$$

controlled by the configurational entropy and the stem cell contact interaction,

$$F [ \{ c \} ] = {k}_{B} T {\sum }_{X} {c}_{X} ln {c}_{X} - \frac{J}{2} [ {c}_{S}^{2} - {α ( ∇ {c}_{S} )}^{2} ] .$$

Here J/k_{B}T is a dimensionless constant characterising the strength of the stem cell adhesion and α is a dimensionless constant of order unity. The last term in (1) describes the division, differentiation and exit of cells from the basal layer with R_{S}=(γ*_{SS}−γ_{SA})c_{S}, R_{A}=γ_{SA}c_{S}−(γ*_{BB}−γ*_{AA})c_{A}, R_{B}=(γ*_{AB}+γ*_{BB})c_{A}−γ_{BΦ}c_{B}, and R_{Φ}+R_{S}+R_{A}+R_{B}=0, where γ*_{XY}=zc_{Φ}γ_{XY} denote the effective division rates for a lattice coordination z. In the absence of cell division or differentiation (R_{X}=0), Eq. (1) recovers the familiar nonlinear Cahn-Hilliard equation describing macroscopic phase separation through spinodal decomposition (A. J. Bray, Advances in Physics, 51, 481 (2002)). Restoring the “reaction” processes, R_{X}, a linear stability analysis shows that, apart from the pathological “jammed” or “empty” states (c_{A}(r)=1 or c_{Φ}(r)=1), the uniform system is unstable towards pattern formation. A numerical integration of the coupled nonlinear equations reveals that the stationary solution is characterised by a regular hexagonal array of dense stem cell clusters within a sea of A and B cells, with slow stem cell division concentrated on the cluster edges.

### Inferring Differentiation Rate

Despite the complexity of the nonlinear equations, the general properties of the steady-state solution can be inferred straightforwardly. In particular, one may obtain the dependence of cluster size on the microscopic parameters by noting that, once differentiated, stem cells satisfy a simple diffusion equation within each cluster. By solving this diffusion equation while simultaneously minimising the energy associated with stem-cell adhesion (embodied in the function F defined above) one may show that the average number of stem cells within each cluster is inversely proportional to the differentiation rate γ_{SA}

$${N}_{S} ∝ \frac{σ}{{γ}_{SA}} exp [ - J / 2 {k}_{B} T ] .$$

Inferring Size and/or Separation of Stem Cell Clusters

Secondly, since, for each stem cell cluster and its associated surroundings, the cell production rate matches the exit rate through differentiation or migration, one obtains the following relations for the steady-state distributions,

$$\frac{{φ}_{A}}{{φ}_{S}} = \frac{{γ}_{SA}}{{γ}_{BB}^{*} - {γ}_{AA}^{*}} , \frac{{φ}_{B}}{{φ}_{A}} = \frac{{γ}_{AB}^{*} + 2 {γ}_{BB}^{*}}{{γ}_{B Φ}}$$

where φ_{S}, φ_{A}, and φ_{B} denote the volume fraction of stem, CP and post-mitotic cells in the basal layer and the rate constants γ*_{XY}=zc_{Φ}γ_{XY} are evaluated for the hole concentration c_{Φ} evaluated in the stem cell depleted region. From these results, the size and separation of the stem cell rich clusters is readily inferred.

### Suitable Inputs

One of the key advantages of the invention is the extremely flexible manner in which the model may be used to make predictions and inferences about cell behaviour. With this in mind, it is an advantage of the invention that a number of different alternative inputs may be used in order to make a corresponding range of individual predictions or descriptions of the cell population being examined.

Suitably, the stem cell fraction may be determined.

Suitably, the progenitor cell fraction (committed progenitor cell or CPC fraction) may be determined.

Suitably, the post mitotic cell fraction may be determined.

Most suitably, all three cell fractions may be determined, namely the post mitotic fraction, the proliferating fraction and the stem cell fraction.

Typically, the model described herein is based on the presence of these three cell fractions. Therefore, typically, the sum of these fractions is to be regarded as 100% of the cell population. Therefore, by determining any two of those fractions, the size of the third fraction is also determined as the balance of the population made up to 100%. Of course, independent determination of each of the three fractions may be used in order to maximise accuracy.

Suitably, the size of stem cell cluster may be determined. The mode by which this is determined is a matter of choice for the operator as discussed below.

Suitably, the distance between clusters may be determined (this may be determined as the lattice period or may be determined as the wave length of clustering, or may simply be determined as the mean distance between adjacent clusters).

Another way in which the number of stem cells per cluster may be estimated is to measure the number of cells per lattice period. The “lattice period” can be regarded in simpler terms as the “wave length” of clustering. In other words, once cells have organised themselves into the characteristic regular pattern, this pattern can be seen to repeat or to be reproduced at regular intervals. Thus, the number of cells such as the number of stem cells per repeat period (lattice period/wave length of clustering) can be conveniently used as an estimate of the number of cells per cluster, or as an estimate of the proportion of stem cells in the overall population.

It must be borne in mind throughout that if a population of cells has been so affected that no clusters or patterning are observed, then this is an indication that the regulation or behaviour has been disrupted to such a large degree that further meaningful analysis may not be possible. In the context of drug testing, if a candidate drug produces the effect of disrupting regulation to the point where no patterning or no clusters are observed, this would typically be taken as a strong indication that that candidate drug has adverse bioactive properties and is unlikely to represent a substance safe for human or animal use.

It is a key advantage of the invention that it may be readily applied to the analysis of human cells. In particular, it may be applied to the analysis of primary human cells cultured according to techniques known in the art. As set out in the example section, it is possible to grow sheets of primary human cells, stain them for the particular markers of cell types which are to be determined, and infer valuable information about the underlying biological processes by means of that analysis.

Analysis of visualised cells may be conducted by eye. Cells may be counted, areas may be measured, cluster sizes may be estimated, or any other data collection may be performed by the operator. Clearly, it is desirable to automate data collection wherever possible in order to streamline or optimise the process, for example to speed it up or to save labour. Any of the readily available commercial tools for automation of image collection or image analysis may be applied in this context. For example, standard staining protocol can be used, the cells are thereby stained for the particular markers being used to indicate their particular proliferative or other state, the cells may be examined using a confocal microscope, and a standard image capture program associated with the confocal microscope may be used to harvest or capture the images. Typically, the operator will determine the number of images required for any particular process, however, it, may be convenient to take approximately 20 to 200 images for a given analysis. These images many then be used manually or in an automated process in order to make the measurements desired for the particular analysis, for example to measure the stem cell cluster sizes.

### Applications

It should be apparent to the skilled reader that the invention may be advantageously applied at several overall layers of analysis. In other words, the invention may be applied to provide different depths or levels of detail depending on the needs of the operator.

Firstly, on a high level, the techniques may be applied in order to check for the presence or absence of patterning (e.g. a loss of patterning). As noted above, if patterning is so disrupted as to be difficult to detect, or if it is lost altogether, it should be understood as a compelling indication that the treatment or conditions applied to those cells has profound effects and is unlikely to be suitable for application to animals or humans.

On a second level, in slightly more detail, it is possible to observe adjusted or altered patterning created by particular treatment or conditions. This would be a strong indication that the particular treatment or condition affecting the cells was altering their regulation in some manner. For many purposes, this would be sufficient information. For example, this observation alone tells the operator that there is a significant biological effect taking place, but that it is not so strong or severe as to have totally abolished patterning itself. Whether or not further detailed analysis is required is a matter of choice for the skilled user.

Thirdly, the invention renders it possible to study the detail of adjustment of the particular biological processes affecting the patterning. For example, observing differences in clone size distributions (cluster sizes) may indicate a change in a rate of cell division, a change in a rate of differentiation, or a migration effect. However, if it is desired to pinpoint the precise biological process which has been perturbed by particular condition or treatment, then the invention may be applied to provide a greater level of detail. For example, in order to investigate whether or not the migration rate has been altered, the population aggregation experiment described above may be performed (plating out a labelled enriched stem cell population together with an unlabelled stem cell depleted population of cells in a homogeneous manner, and following the speed with which patterning is re-established). A faster or slower time to re-establishment of patterning would indicate an increased or decreased migration route respectively. Alternatively, if it desired to understand whether it is an effect on cell division rate, single cell seeding experiments or clone analysis may be undertaken in order to confirm or eliminate that possibility. Further, more detailed analyses are described herein and may be deployed by the skilled operator according to their preferences or the application to which the invention is being put.

Thus, it is clear that the invention is extremely adaptable. For example, the invention may be applied in the format of a high throughput screen collecting a limited amount of information about a range of different treatments. In this embodiment, the invention might be applied to simply score for the loss or retention of patterning, providing almost binary output for the particular treatment or conditions applied. On an intermediate level, it may be desired to deploy the invention to understand how patterning has been affected by a particular treatment or condition. In this embodiment, it may be desirable to score for various different types of adjusted patterning rather than a mere binary score of whether or not patterning has been affected. However, equally, in this embodiment it may not be desired to discern further information about the particular processes which have been analysed. In another embodiment, the invention may be used to investigate and pinpoint exactly which of the candidate biological processes has been perturbed by a particular condition or treatment in order to provide a detailed understanding of the biological consequences in a particular setting. It is this adaptability which is a key, advantage and benefit of the present invention.

### Cells

Suitably the invention may be applied to any mammalian cells. More suitably the invention may be applied to human cells. Most suitably the invention may be applied to human epidermal cells, most suitably the invention may be applied to human keratinocytes.

### Organotypic Culture

A number of cell/tissue systems are capable of self-organisation in vitro. This means that tissues or tissue-like structures can be cultured in vitro. This is termed organotypic culture. One example of this is epidermis. For example, a preparation of stem cell keratinocytes (e.g. MCSP positive cells) can be plated out and cultured. These will then self organise into epidermis which is a fully three-dimensionally organised, organotypic stem cell clustered structure which persists for at least about 30 days in vitro. Suitably the invention is applied to organotypic cultures. The advantage of this is excellent reproducibility, reduced need for animals or biopsy samples of human cells, and use of a tractable system amenable to in vitro handling. Such systems are known in the art, such as the ‘Skinethic™’ system from L'Oreal which is an ISO 9001 compliant ‘off-the-shelf’ system suitable for use in the present invention. Thus in one embodiment the cells are suitably keratinocytes in the form of an organotypic culture such as an organotypic keratinocyte culture. This has the advantage of being an excellent model closely tracking the situation in vivo. This has the further advantage of permitting analysis and study of primary mammalian cells derived directly from a subject. This advantageously avoids possible problems or complications arising from use of long-term or immortalised cultured cells which, whilst useful, can be considered less closely connected to the actual situation in vivo. Organotypic culture is particularly suitable for epidermis, oesophagus, trachea, cervix, oral mucosa, bladder, thymus, brain, spinal cord (e.g. by slice cultures, can be transfected/microinjected/infected with a viral vector to track cell fate), cancer (various, including squamous carcinomas, breast carcinoma), adipose tissue and bone marrow.

It is preferred that the invention does not involve the actual steps of removing cells from the animal or human body. Typically the invention is carried out using cell cultures in vitro ie. cultures which have been established in the laboratory or clinical setting. Therefore methods of the invention are suitably in vitro methods. Methods of the invention suitably do not involve the actual step of biopsy or cell collection from a human or animal subject. If it is desired, the step of cell collection may be included in the methods of the invention according to operator choice, but preferably such a step is omitted and the invention is practiced using established cultures in vitro.

Suitably the cells are human cells.

It should be noted that although stem cells are discussed herein, suitably these stem cells are not totipotent ie. they cannot give rise to every possible tissue type and as such cannot even theoretically be considered to give rise to a human or animal or embryo. Indeed, as will be apparent from the specification, suitably the stem cells examined are already lineage restricted but capable of self renewal and generating committed progenitor cells that will all ultimately become terminally differentiated cells (‘post-mitotic’ cells). Therefore the stem cells mentioned are suitably epidermal stem cells (which are not totipotent), most suitably human epidermal stem cells.

It is an advantage of the invention that the cell culture techniques used are entirely standard and well known in the art. Working with the invention requires no special equipment or irregular culture techniques beyond those already well known to the skilled operator. In particular, suitably cells may be cultured according to methods noted in example 3.

### Combination Applications

Suitably the method of the invention may be combined in a dual approach with clonal analysis i.e. analysis of clone size distribution. Such analysis is known in the art but in case any further guidance is needed, the following should be noted:

### Clonal Analysis/Clone Size Distribution

Suitably clonal analysis comprises analysis of clone size distribution and is carried out as is known in the art, such as taught in WO2007/101979 (PCT application number PCT/GB2007/000675) by the same inventors.

The model of cell growth behaviour which is used to predict the value of the proliferation characteristic (clone size distribution) for normal cells may be defined by the parameters of: the overall division rate of cycling cells (λ); the probability that the division is asymmetric (p_{AD}); and the rate of transfer of non cycling cells from the basal to the suprabasal layer (Γ).

Assuming that the rates of symmetric cell division to cycling and non-cycling daughter cells are identical for normal cells, these parameters may then be related by the equation:

$$\frac{d }{d t} {P}_{mn} = λ \{ \frac{1}{2} ( 1 - {p}_{AD} ) [ ( m - 1 ) {P}_{m - 1 , n} + ( m + 1 ) {P}_{m + 1 , n - 2} ] + {p}_{AD} {mP}_{mn - 1} - mP + Γ [ ( n + 1 ) {P}_{mn + 1} - {nP}_{mn} ]$$

where Pₘₙ(t) is the probability that the cells consist of m cycling cells and n non-cycling cells after a time t after induction and, Pₘₙ(0)=nδₘ₁δₙ₀+(1−n) δₘ₀δₙ₁.

In some embodiments, changes in the cell growth behaviour may include changes in the ratio of λ_{AA}:λ_{BB}. For example, whilst in normal cell proliferation and differentiation, λ_{AA}:λ_{BB} may equal 1, λ_{AA}:λ_{BB} may be found to be greater than 1 or less than 1 in the target cells. This may be indicative of either excessive proliferation, which may, for example, be indicative of cancer, or proliferation which may be insufficient for tissue homeostasis.

The same equation may be conveniently written in the following form: Defining Pₙ_{_{A}}_{,n}_{_{B}}(t) as the probability that a labelled clone involves n_{A} A-type and n_{B} B-type EPCs at time t after induction, its time-evolution is governed by the Master Equation:

$$\frac{d {P}_{{n}_{A} , {n}_{B}}}{d t} = λ \{ r [ ( {n}_{A} - 1 ) {P}_{{n}_{A} - 1 , {n}_{B}} + ( {n}_{A} + 1 ) {P}_{{n}_{A} + 1 , {n}_{B} - 2} ] + ( 1 - 2 r ) {n}_{A} {P}_{{n}_{A} , {n}_{B} - 1} - {n}_{A} {P}_{{n}_{A} , {n}_{B}} \} + Γ [ ( {n}_{B} + 1 ) {P}_{{n}_{A} , {n}_{B} + 1} - {n}_{B} {P}_{{n}_{A} , {n}_{B}} ]$$

subject to the initial condition Pₙ_{_{A}}_{,n}_{_{B}}(0)=ρδₙ_{_{A}}_{,1}δₙ_{_{B}}_{,0}+(1−ρ) δₙ_{_{A}}_{,0}δₙ_{_{B}}_{,1}.

This is clearly the same as the equation presented above, with (1−2r)=P_{AD} and with n_{A}=m and with n_{B}=n and thus n_{A},n_{B}=m,n. For convenience the methods of the invention preferably refer to this ‘Master Equation’.

Values for these or other, alternative parameters may be determined by fitting the model to data derived for normal cell growth behaviour (i.e. cells which proliferate and differentiate normally). For example, a method may comprise measuring the values of one or more proliferation characteristics of normal cells, preferably two or more proliferation characteristics, and fitting the parameters to the measured values.

Further combination applications may include:

Performance of clonal density (in vitro clonal analysis) to get indication of stem cell/committed progenitor cell ratio; and then performing a method according to the present invention to detect an altered behaviour. Suitably the method of the invention is an embodiment using organotypic culture based patterning analysis.

Optionally the method may further comprise validating in vivo by carrying out clone size distribution analysis as set out above.

### Further Applications

The invention also enables experimental approaches to reveal stem and committed progenitor cell fate and pattern formation.

Suitably said population of cells is a population of epithelial cells such as mammalian epithelial cells. Although the invention has been described with reference to mammalian epidermal cells as a model epithelial system, it will be appreciated that the invention may be applied to other similar cells eg. from other stratified squamous epithelia such as oesophagus, oral cavity, cervix and similar tissues. Such cells exhibit similar stem cell clustering to those exemplified for skin and therefore any tissue system exhibiting such patterns is suitable for use or study herein.

The invention finds application for example in high throughput screening to identify conditions, chemical agents, treatments or the like which affect behaviour within a population of cells, in particular which affect behaviour of subset(s) of cells within the population. Moreover the invention finds application in the dissection and study of cell behaviours eg. differentiation rate, division rate, migration rate or other such property including cell to cell adhesion capacity. The level of detail of analysis is a matter for operator choice as illustrated herein.

The populations of cells may be subjected to any treatment of interest. Many of the embodiments described related to addition of chemical entities such as candidate drugs or pharmacologicl agents. However, it should be borne in mind that the first and second populations of cells may be instead be cells transfected with experimental or control siRNA, or may be cells expressing an experimental or control gene or silencing cassette; or cells to which any other treatment has been applied together with an appropriate reference or control population.

Clearly the reference values may not need to be generated by parallel handling and analysis of a second population of cells each time a first population of cells is treated. For reproducible systems, the reference values are advantageously simply determined once and then used each time for comparison.

Elements of the method(s) of the invention may be automated for example to apply the methods to high throughput screening. For example, simple software may be applied to identify/outline clusters and count cells. Such automation is entirely within the capacity of the person skilled in the art. Indeed, this can already be carried out by software such as the ‘Velocity™’ software to collect inputs for the model(s) of the invention.

An interesting application of the model presented herein is in understanding the behaviour of cells in the absence of cell division. As noted above, the inventors have surprisingly shown that the model conforms to a spinodal decomposition. In other words, the model predicts the clustering should occur with no need for cell division to be taking place. Indeed, this can be experimentally demonstrated. A population of keratinocytes is produced. These are each labelled with a visualisable marker. This population is then enriched for stem cells. This stem cell enrichment may be easily accomplished by plating the cells on collagen. The stem cells attach more readily to the collagen, and therefore by washing away unattached cells a population of cells enriched for stem cells is produced. This stem cell enriched population can then be overlaid with unlabelled stem cell depleted cells. Thus, overall this reconstitutes a population of cells equivalent to the starting population, but where the majority of the stem cells are labelled and therefore their arrangement can be, visualised. Upon observing this system, it is seen that a lot of cellular movement takes place for approximately 18 hours following constitution of the cell population. At this point visualisable red patches clearly emerge as a pattern from the previously uniform cellular background. In other words, stem cell aggregation is taking place. It is absolutely clear that this system does not depend on cell division, since 18 hours is a time frame which is too short to permit any significant cell division. Thus, it is experimentally demonstrated that one of the key predictions of the model is indeed borne out by the behaviour of the actual cell populations.

It is a feature of the model that any stem cell in a cluster can differentiate. Differentiated cells are no longer stem cells, and therefore are less likely to aggregate with stem cells. Furthermore, differentiated cells in an epidermal setting migrate out of the basal layer into the supra basal layer and therefore leave the clusters of their own accord. Clearly, if the differentiation rate was too high, the clusters would “fall apart” eg. disaggregate or would struggle to form at all. Therefore, it can be clearly appreciated that the cluster size has a relationship to the differentiation rate of stem cells therein. Thus, one application of the model of the invention is to use information about the cluster sizes to infer information about the differentiation rate. On a gross level, this enables simple determination such as a measurement of the stem cell fraction to yield information about a higher biological process such as a cell division rate or a cell differentiation rate.

The invention may be usefully applied to the study of cancer. For example, pathological tissue may be cultured and studied according to the present invention. In another embodiment, clonal labelling may be undertaken, for example by preparing a culture from a population of MCSP positive cells which population comprises an introduced sub-population of labelled cells such as cells expressing green fluorescent protein. Moreover, cells from a cancer cell line may be added into an organotypic culture system and their fates followed. Thus in some embodiments the population of cells comprises cancer cells. In some embodiments the population of cells may comprise one or more labelled clones of cells.

In some populations of cells studied by the present invention, the cells may initially be plated out at ‘clonal density’ which refers to a sparse plating of cells such that they are essentially dividing individually during the initial phase of proliferation. This can have the effect of accelerating the rate of division. FIG. 9C illustrates observations from this application of the invention. FIG. 9 overall shows an excellent quantitative fit demonstrating that the stochastic fate of the cells is hard wired and not determined by environmental cues in this system—this is discussed in more detail in the examples section.

Many of the examples presented feature unsorted cell populations. However, it will be apparent to the skilled worker that sorted cells may equally be used. For example, the population of cells being studied may comprise unsorted keratinocytes, or may comprise flow sorted cells bearing (or not bearing) a particular marker such as a stem cell marker. Moreover, cells may be sorted by other techniques such as by plating for about 15-20 minutes and washing the cells when stem cells will stick, non-stem cells will be washed off. Other variants of such techniques will be apparent to the skilled worker, such as by plating on collagen for about 60 minutes to remove stem cells by washing off and using the non-stem cell population which do not stick in that time frame. Such sorting techniques have the advantage of adding an extra layer of control and/or information to the analysis being conducted. Clearly, depending on the starting population, the more adherent and less adherent cell populations may not strictly correspond to stem and non-stem cells—the operator will determine the nature of the different fractions.

Further applications are described in example 7 and the associated figures.

In preferred embodiments, the reference values are described or predicted from the model presented herein.

### Mathematical Model

### S-I.1 Classification of Type I and Type II Clones

From earlier studies [4], as well as this one, it is apparent that a subset of cells seeded at clonal density give rise to exponentially expanding clones. However, the quantitative behaviour of the remaining clones is not well understood. Therefore, to characterise the behaviour of the progenitor cells in culture, we shall begin by classifying clones into type I and II sub-populations according to whether or not the clones appear to expand geometrically. Following this classification, we shall analyse the “type I” clone population behaviour for signatures of stochastic cell fate.

In order to distinguish between clones deriving from initially-seeded stem and non-stem cells, it is helpful to first consider the behaviour of the overall clone population, as seen by the average clone size in FIG. 3E. The average done size grows exponentially from approximately 20 hours onwards, with a population doubling time of 20±0.8 hours (standard error obtained from regression analysis). Such a “shock time”, in which cells do not divide for an initial period post-plating, has also been observed in other systems [5].

To identify non-stem cell derived clones, we looked for a sub-population of clones that were significantly smaller than expected from the overall average size. Such clones may arise from cells that either divide significantly more slowly than the average, or that are unable to divide exponentially. As a rule of thumb, clones whose size corresponded to a population doubling time longer than twice the average were designated as non-stem cell-derived, whereas clones whose size corresponded to a population doubling time shorter than or equal to the average were designated as stem cell-derived. At times earlier than 72 hours, the differences in population doubling time are difficult to discern (as all exponents behave linearly at short times). We therefore concentrated on the later time points (72-168 hours), where the exponential growth of stem cell-derived clones would reduce errors in classification. As shown in FIGS. 3B and S2, clones of size smaller than or equal to 8, 16 and 32 cells were classified as type I clones at 72, 96 and 168 hours post-seeding, respectively, and, at the same time points, clones larger than 32, 64 and 128 were classified as type II clones. To further identify stem cell-derived clones, the cell growth marker Ki67 was then used to assess the fraction of cycling cells for the remaining unclassified clones. Clones in which over 50% of cells were Ki67-positive were classified as stem cell-derived, while the remaining clones were classified as non-stem cell-derived, see FIGS. 3B and S2.

As some time may be required for the loss of Ki67 immunostaining following differentiation, there is a question about reliability of Ki67 as a real-time label of cycling cells. It is safe to assume that the number of Ki67-positive cells presents an upper limit to the overall number of cycling cells within a clone. Allowing for such a time delay before loss of Ki67 immunostaining in a newly-formed post-mitotic cell, it would be a significant challenge to distinguish between proliferating and differentiating clones during the early hours past-seeding. This motivates attempting a segregation

FIG. S1: The full clone size distribution data used to generate FIG. 3A. The number of clones scored at each time point lies in the range of N=325-525 clones. Error bars show SEM.

of the data only at later time points, where the impact of such effects in minimal.

Although these rules provide a reliable classification of clones with 3 or more cells, there is some uncertainty surrounding the contributions to the 2-cell clone population. Despite their consistent classification as type I clones, the possibility of direct (stochastic) differentiation of stem cells may lead to a population of terminally differentiated two-cell clones of stem cell origin. For example, with a stem cell differentiation rate estimated at 50% of the symmetric division rate (see below), one would expect fully one third of all stem cells to result in clones committed to terminal differentiation. While one could proceed by excluding all two-cell clones from further analysis, here we have taken a different approach: using a quantitative stochastic model to fit the size distribution of type I clones (see section S-I.4 below), we were able to estimate the size of the type I 2-cell population at later times. The remaining two-cell clones were assumed to result from stem cell origin, and were therefore transferred to the type II population as shown by the green and red concentric data points in FIGS. 3B and S2. This classification provides an estimate for the significance of direct stem cell differentiation in culture.

FIG. S2: Classification of clones according to stem cell and non-stem cell origin at 72 and 96 hours. Clones are classified according to their overall size (horizontal axis) and the number of cells expressing the cell cycle marketer Ki67 (vertical axis). The size of the data points indicates the number of clones belonging to each category (see legend). The grey region denotes clones of non-stem cell origin (type I, red), while the remaining area defines clones of stem cell origin (type II, green). To account for direct differentiation of stem cells, a proportion of the differentiated two-cell clones is assigned to stem cell origin as discussed in the supplementary text.

### S-I.2 Quantitative Analysis of Stochastic Cell Behaviour

To quantify the stochastic behaviour of cells from the full clone size distribution, we shall orient our analysis around a stochastic birth-death process consisting of stem (S), committed progenitor (CP) and post-mitotic cells. As per the main text, we will consider stem cells that self-renew through symmetric division and differentiate into committed progenitor cells. However, due to the importance of environmental regulation of stem cells, as seen by their quiescence in confluent cultures (FIG. 2B) and by the change in clonal growth rates after 7 days in vitro [6], we will not attempt to model the full behaviour of stem cells in culture. Denoting CP cells by “A” as in the main text, we focus instead on the basic features of self-renewal and differentiation, viz.

$$\begin{matrix}S {\to }^{{λ}_{S}} S + S \\ S {\to }^{{γ}_{A}^{( S )}} A ,\end{matrix} \qquad ( S1 )$$

where the stem cell division and differentiation rates are, respectively, λ_{S} and γ_{A}^{(S)}. By contrast, for CP cells, which are the focus of this analysis, we shall consider the complete set of processes consistent with the observed two-cell clone fate (FIG. 3D), by which each CP cell (A) may either divide symmetrically or asymmetrically into a CP and post-mitotic (B) cell, according to the following rules:

$$A {\to }^{{λ}_{A}} \{ \begin{matrix}A + A & Prob . r - ε \\ A + B & Prob . 1 - 2 r \\ B + B & Prob . r + ε .\end{matrix} \qquad ( S2 )$$

The average CP cell division rate is λ_{A}, and the “branching ratios” between the different channels of CP cell fate are denoted by r±ε. Thus, r+ε denotes the probability of a CP cells dividing in vitro to give rise to two daughter CP cells, and so on for the remaining channels shown in (S2). The imbalance ε between the rates of symmetric CP cell division controls the lifetime of CP cell-derived clones: a large (positive) value of ε implies that CP cells are rapidly driven towards terminal differentiation. FIG. 3B shows that type I clones continue to proliferate throughout the duration of the experiment, implying that the imbalance, if present, is small (ε<<1), and beyond the resolution of the experimental data. Therefore, in the following we will suppose that ε=0.

### S-I.3 Analysis of Average Size of Type I and Type II Clones

Denoting the average number of stem, CP and post-mitotic cells per clone as (n_{S}), (n_{A}) and (n_{B}), and the total number of cells per clone as (n)=(n_{S})+(n_{A})+(n_{B}), the dynamical equations associated with processes (S1-S2) are:

$$\frac{d 〈 {n}_{S} 〉}{d t} = ( {λ}_{S} - {γ}_{A}^{( S )} ) 〈 {n}_{S} 〉$$

$$\frac{d 〈 {n}_{A} 〉}{d t} = {γ}_{A}^{( S )} 〈 {n}_{S} 〉$$

$$\frac{d 〈 {n}_{B} 〉}{d t} = {λ}_{A} 〈 {n}_{A} 〉 .$$

From these equations, one may calculate the average size of clones derived from a single stem or CP cell. For a single stem cell seeded at time t=0, the average clone size is

$$〈 n ( t ) 〉 = 1 + ( 1 - \frac{{λ}_{S}}{{λ}_{S} - {γ}_{A}^{( S )}} ) {λ}_{A} t + \frac{{λ}_{S}^{2} + {γ}_{A}^{( S )} ( {λ}_{A} - {λ}_{S} )}{{( {λ}_{S} - {γ}_{A}^{( S )} )}^{2}} ( {e}^{( λ - {γ}_{A}^{( S )} ) t} - 1 ) . \qquad ( S3 )$$

For an initially seeded CP cell, the average clone size is (n(t))=1+λ_{A}t. With these expressions, we note that linear growth in the average type I clone size in FIG. 3E is consistent with CP cell behaviour, while the exponential growth of type II clones is consistent with stem cell behaviour.

A linear fit to the average type I clone size (FIG. 3E) translates to a division rate of λ=2.4±0.7/day (standard error obtained from regression analysis) for CP cells, or an average CP cell cycle time of 10±2 hours. For type II clones, a fit of the average to Eq. (S3) is consistent with an exponential growth rate of λ−γ_{A}^{(S)}=0.79/day, or a population doubling time of 21 hours. An independent fit of the division an differentiation rates is consistent with λ=1.6/day λ−γ_{A}^{(S)}/λ=50%, however a range of division rates (λ=1.6-2.4/day) provide a reasonable fit to the data, provided that the differentiation rate is chosen appropriately to maintain the same exponential growth rate.

The flexibility in the fit to the type II average clone size data makes it difficult to quantify the division and differentiation rates associated with the type II clones. However, their observed exponential growth, together with the appearance of Ki67-negative cells, indicate that they originate from stem cells capable of self renewal and differentiation. While the rates of division and differentiation in culture remain interesting in their own right, it is doubtful whether these rates are simply related to the behaviour of stem cells in vivo, the focus of this study. Therefore, in the following we shall focus only on the properties of the CP cell population, where the detailed stochastic behaviour is of particular interest by virtue of its similarity to that seen in murine epidermis in vivo.

### BRIEF DESCRIPTION OF THE FIGURES

FIG. 1 shows a quantitative model of tissue maintenance in interfollicular epidermis.

FIG. 2 shows a hydrodynamic description of the average cell densities.

FIGS. 3 to 6 show flow charts of methods.

FIG. 7 shows three diagrams. FIGS. 7A and 7B show comparative diagrams helpful in understanding the art; in FIG. 7C-K is keratinocyte, S is stem, CP is committed progenitor and PM is post mitotic. In particular FIG. 7 shows structure of the epidermis, and the stem/CP cell hypothesis

(A) Schematic showing the architecture of mammalian epidermis. Stem cells located in the bulge (b, green) maintain the hair follicle. The organization of keratinocytes in interfollicular epidermis (IFE) is shown inset. Proliferation is confined to the basal layer; cells migrate out of the basal layer as they differentiate, eventually being shed at the skin surface (arrows).

(B) Stem/TA cell hypothesis: Slow-cycling stem cells (S) give rise to a short-lived population of transit-amplifying (TA) cells that undergo 3-5 rounds of division before terminal differentiation into post-mitotic (PM) cells.

(C) Stem/CP cell hypothesis: Stem cells (S) divide to give rise to two daughter stem cells, or differentiate into a committed progenitor (CP) cell. CP cells give rise to either two daughter CP cells, or one CP cell and one post-mitotic cell, or two post-mitotic cells. Both stem and CP cell division is inhibited by the accumulation of local cell density, as denoted schematically by the shaded neighbouring cell that may be any type of basal layer keratinocyte (K). While CP cell fate remains stochastic, stem cells respond to a drop in the local cell density by becoming biased towards self-renewal.

FIG. 8 shows photomicrographs and a diagram. FIGS. 8A and B show human breast epidermis; green (clusters) are stem cells; red (dots) are cycling cells. Proliferation within the clusters is rare. Cycling cells are mostly on the edges or in between clusters. This is an illustration of a key concept of the invention that patterning is an intrinsic property of the stem cell compartment. In particular FIG. 8 shows In vivo and in vitro patterning and quiescence of stem cells.

(A) Rendered confocal Z-stack of a human breast epidermis wholemount stained for the stem cell marker MCSP (green) and the cell cycle marker Ki67 (red). The staining reveals an irregular pattern of quiescent stem cell clusters surrounded by a sea of non-stem progenitor and post-mitotic cells. The outline of two typical MCSP-bright patches is indicated. (See supplementary for movie.)

(B) Rendered confocal Z-stack of an organotypic culture stained as in (A).

Lower panels show cross sections along the dashed lines in (A, B), with the basal layer outlined. DAPI is shown in blue. RR=rete ridge, DP=dermal papilla. Scale bars=100 μm.

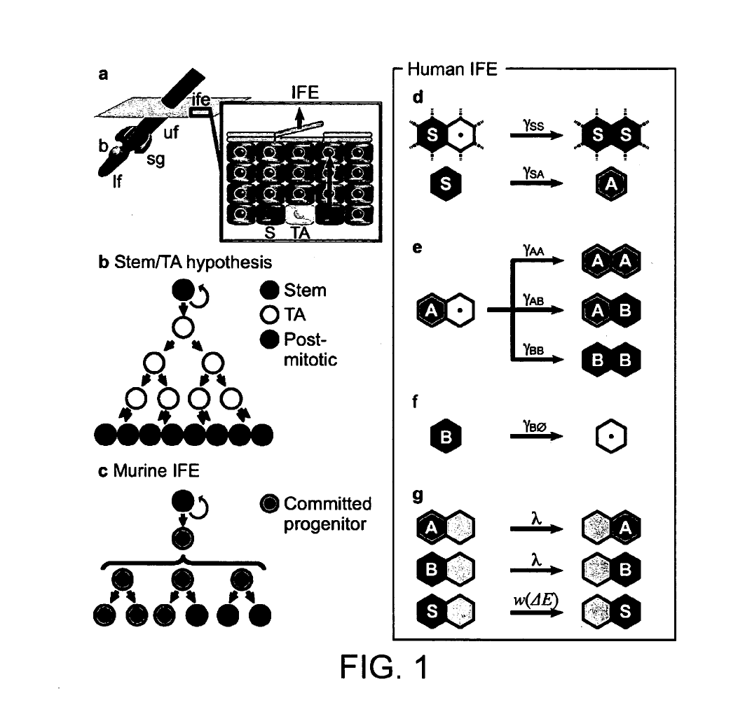

(C) Typical realisation of the stem/CP cell model (FIG. 1C) obtained through a cellular automata simulation (see supplementary section S-V). Clusters of stem cells (green) are seen within a sea of CP (red) and post-mitotic cells (grey).

FIG. 9 shows clonal analysis of human keratinocytes in vitro

(A) Size distribution of clones of unicellular origin at 24-168 hours post-seeding, from a sample of at least n=325 clones per time point (error bars indicate SEM).

(B) Classification of clones according to their overall size and level of Ki67 expression at 7 days post-seeding. The axes show the number of cells per clone grouped in increasing powers of two, e.g. the circle at coordinate (6,4) shows that 5 clones had a total size in the range 33-64 cells of which 9-16 were Ki67-bright. The bars indicate that, in very large clones, at least 25% of cells are Ki67 bright. The grey region defines type I clones (red) with the remaining region of type II (green) (see methods for classification). Note that terminal differentiation in both type I and type II clones may contribute significantly to the appearance of Ki67-dull two-cell clones (see methods and supplementary section II).

(C) Visualisation of colonies at 72 hours post-seeding demonstrating the variation between a type II clone (left) and two type I clones (centre and right). Cells are stained for DAPI (blue), keratin 14 (green), and Ki67 (red). Feeder cells may be distinguished from keratinocytes by the absence of keratin expression. Scale bar=50 μm.

(D) Visualization of two-cell clones showing the three possible proliferative fates of the daughter cells from a single division. Clones are stained as in (C). Scale bar=20 μm.

(E) Data points show the average number of cells per clone over the time course shown for all multi-cellular clones (black), and separately for the sub-populations of type I (red) and type II (green) clones. Cell division is suppressed for approximately 20 hours post-seeding. Lines show the results of theory for which type I clones are derived from CP cells with a division rate of 2.4/day, and type II clones are derived from stem cells with a division rate of 1.6/day and a differentiation rate of 0.8/day (solid green), see supplementary S-I for details. For comparison, the dashed green line shows a fit to a simple exponential corresponding to stem cell division without differentiation. Noting that 60% of clones belong to the type I population and the remaining 40% to type II, one can use these fits to infer the average clone size of the entire population (black curve).

(F,G) Fit to the clone size distributions for all type I clones (F), and for the type I sub-population of fully-differentiated clones (G) (error bars indicate SEM). Circles show the results of theory obtained by assuming that the type I clones derive from CP cells that obey the rules shown in (H) (see supplementary section S-I).

(H) The stochastic process of CP cell division/differentiation used to fit the experimental data shown in (F,G).

FIG. 10 shows spontaneous regulation of stem cell cluster size

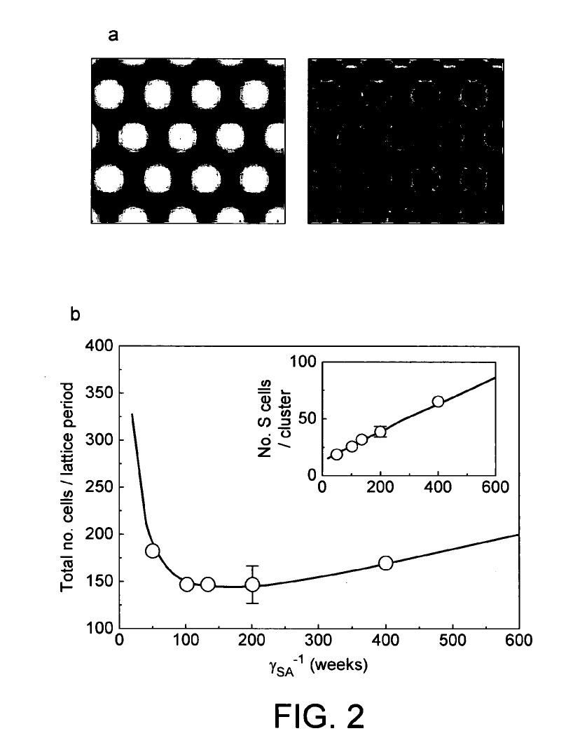

(A-D) Fragmentation of an oversized stem cell cluster evolving according to the rules of the stem/CP cell model (FIG. 1C) as obtained through a cellular automata simulation (see supplementary section S-V). Cell colours as in FIG. 2C. Panels refer to time points as shown.

FIG. 11 shows behaviour of the stem/CP cell model

(A) Stationary stem cell density as obtained from the numerical solution of Eq. (1).

Stem cells aggregate into a periodic array of clusters (green), within a sea of committed progenitor and post-mitotic cells (pink). The regularity of the pattern is due to the “mean-field” character of the theory, which describes the average behaviour of the population (see main text). The location of stem cells in cycle (black) (as measured by the product c_{S}(r,t) γ^{(S)}_{SS}(r,t)) is shown inset for the same stationary state, revealing that stem cells deep within each cluster are quiescent. The lower panel shows the density of stem (type S), committed progenitor (type A) and post-mitotic (type B) cells along the dashed cross-section.

(B) Theory curves indicating the dependence of the steady-state stem cell (green) and CP cell (red) volume fractions on the stem cell differentiation rate, γ^{(S)}_{A}, in units of the post-mitotic stratification rate, γ^{(B)}. The stem cell differentiation rate is shown to play an important role in determining the volume fractions, as well as the size of stem cell clusters (inset). Data points indicate results of cellular automata simulations of the stem/CP cell model (C,D). Model parameter values are defined in the methods section. (C,D) Realisations of the stem/CP cell model (FIG. 1C) obtained through cellular automata simulations, showing the effect of increasing the stem cell differentiation rate from γ^{(S)}_{A}/γ^{(B)}=0.02 (C) to 0.08 (D).

FIG. 12 shows the balance between stem cell self-renewal and differentiation plays a key role in defining the morphology of clones derived from isolated human epidermal stem cells

(A) Phase-contrast micrograph of typical macroscopic colonies at 12 days post-seeding, showing a large round colony and a small wrinkled colony. Scale bar=2.5 mm.

(B,C) Rendered confocal images of typical boundary regions obtained from a large circular colony (B), and a small wrinkled colony (C), showing cells stained for the stem-cell marker MCSP (green) and the proliferation marker Ki67 (red). Scale bars=100 μm.

(D) Cellular automata simulations of the stem/CP cell model demonstrate the transition from large and circular colony growth to irregular and wrinkled development. The two clones originate from a single stem cell in silico, shown after the same number of cell cycles post-seeding. On the left, all stem cell divisions on the perimeter, give rise to two stem cell daughters whereas, on the right, 10% of divisions give rise to two CP cells. Accumulation of differentiated cells on the rapidly proliferating perimeter obstructs stem cell proliferation and results in irregular growth (supplementary section S-VI).

(E,F) Magnified sections from (D) show stem cells in green, CP cells in red and post-mitotic cells in grey.

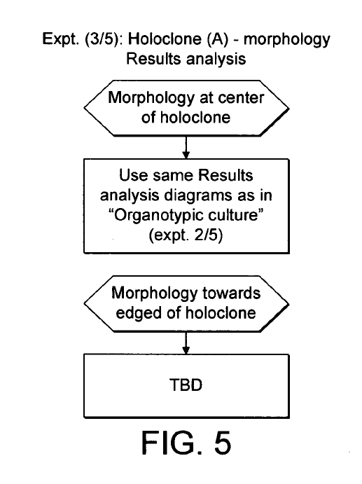

FIG. 13 shows clone size distribution data. FIG. 13 is sometimes referred to as FIG. 5i in the text.

FIG. 14 shows classification of clones. FIG. 14 is sometimes referred to as FIG. S2 in the text.

FIG. 15 shows graphs. FIG. 15 is sometimes referred to as FIG. S9.

FIG. 16 shows examples of predictions made according to the present invention. FIG. 16 is sometimes referred to as FIG. S12.

FIG. 17 shows examples of predictions made according to the present invention. FIG. 17 is sometimes referred to as FIG. S13.

FIG. 18 shows examples of predictions made according to the present invention. FIG. 18 is sometimes referred to as FIG. S14.

The invention is now described by way of example. These examples are intended to be illustrative, and are not intended to limit the appended claims.

### EXAMPLES

### Overview

FIGS. 8A and 8B show cultured cell systems; FIG. 8C shows a diagram of predicted cell behaviour according to the present invention—it can be immediately appreciated that the model is very accurate due to the close match between observation and prediction. FIG. 11 shows the effects of increased rate of stem cell differentiation, which of course alters the ratio of stem cell differentiation to proliferation rate.

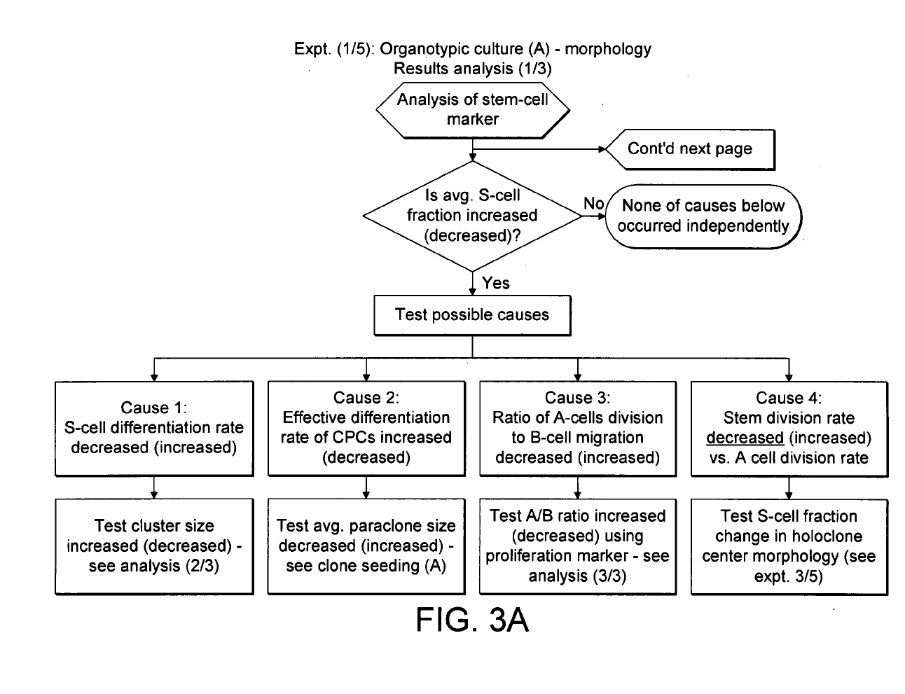

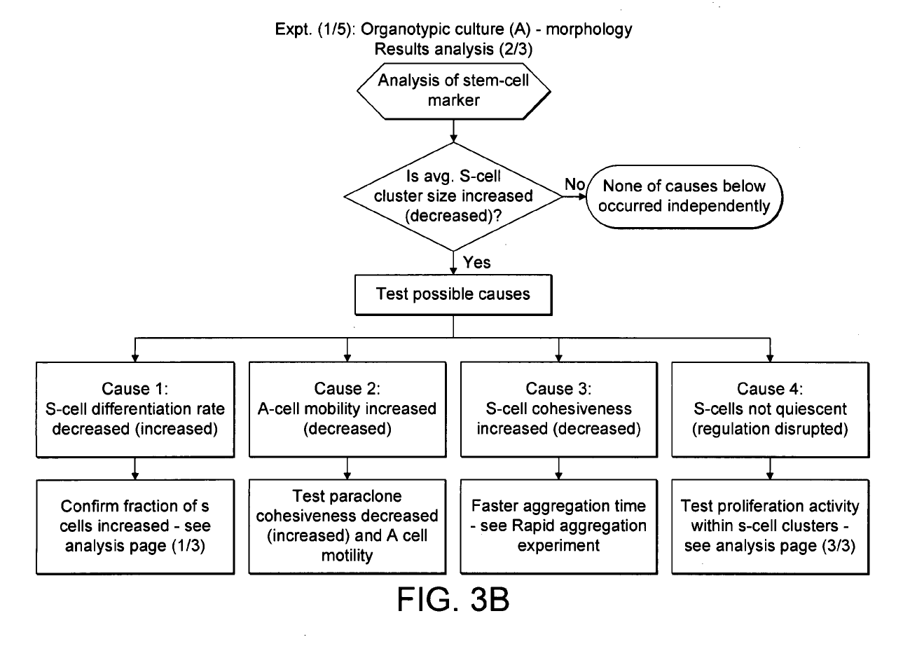

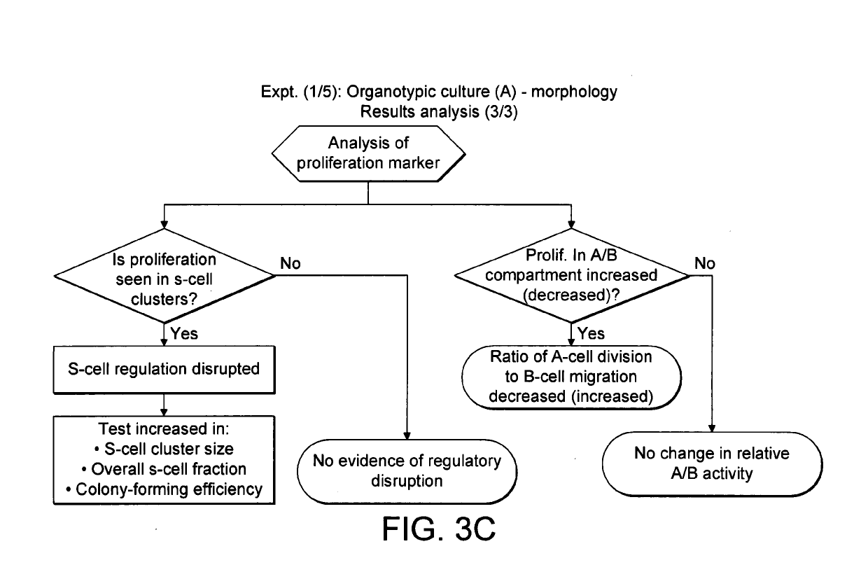

FIGS. 8 and 9 show two different experimental designs which strongly complement each other. FIG. 8 shows an organotypic application of the invention. FIG. 3 shows a clonal density plating experiment. The performance of analysis using both systems is extremely advantageous. Thus in a preferred embodiment a combined analysis is carried out according to the present invention comprising a method of detecting an altered behaviour as described herein, and further comprising performing clonal analysis on the same or equivalent population of cells (see above).

It should be noted that the systems of FIG. 9 may be applied to any cell or tissue system which can be made to remain cohesive in culture. This is especially suitable for non-migratory cell types. In order to apply the invention to migratory cell types (or indeed non-cohesive cells more generally such as non-adherent cells) then cells can be plated into individual wells such as in a 96-well array and incubated. It should be noted that it is a remarkable finding that the channels of cell fate remain unaltered even in such single cell incubations. Indeed, it is the fact that their individual fates/behaviours are preserved in clonal or individual culture that illustrates the powerful techniques of the invention. It should be further noted that if a candidate drug or treatment affects adhesiveness, then this is no barrier to application of the invention since individual culture can simply be employed to overcome this.

FIG. 11 is particularly helpful in illustrating the use of the invention in making predictions regarding cell behaviours. For example, the question can be asked if a parameter changes, then what is the effect? FIG. 11 and other later figures are helpful in demonstrating application of the invention and its utility in predicting changes in cell behaviour in this regard.

### Example 1

### Quantitative Model of Tissue Maintenance in Interfollicular Epidermis

Referring to FIG. 1, FIG. 1a shows a schematic of the architecture of mammalian epidermis. Hair follicles contain stem cells located the bulge (b, green), with the potential to generate lower hair follicle (lf), sebaceous gland (sg, orange), and upper follicle (uf). The inset shows the organization of keratinocytes in interfollicular epidermis (IFE, beige) as proposed by the stem/TA cell hypothesis. The basal layer comprises stem cells (S, blue), transit-amplifying cells (TA, yellow), and post-mitotic basal cells (black), which migrate out of the basal layer as they differentiate (arrows).

FIG. 1b shows the stem/TA hypothesis showing three rounds of cell division in the TA cell compartment.

FIG. 1c shows the stem/CPC model of epidermal maintenance. In mouse tail, stem cells do not contribute to normal epidermal maintenance (grey arrow). Instead, the epidermis is maintained by a single compartment of committed progenitor cells (red) that may divide an unlimited number of times before terminal differentiation.

FIG. 1d-g show extension of this model according to the present invention. This revised and extended model accounts for the existence of a slow-cycling stem cell compartment capable of spontaneous patterning observed in human IFE; we treat the basal layer as an hexagonal lattice of cells. Upon division, stem cells (d) give rise to two daughter stem cells, or they may differentiate into progenitor cells committed to terminal differentiation. Progenitor cells (e) give rise to either two daughter progenitor cells, or one progenitor cell and one post-mitotic cell, or two post-mitotic cells. These, in turn, migrate out of the basal layer towards the skin surface (f), thus creating the capacity for stem cells and committed progenitors to divide into the resulting vacancy (white hexagon). Cells migrate across the basal layer by exchanging places with other cells or with vacancies (denoted collectively in grey, g). The mean rates of cell division in the presence of a vacancy, as well as cell differentiation and migration to the suprabasal layer, are denoted by γ_{XY}. The rate of cell migration is set by the lattice hopping rate λ for type A and B cells; for stem cells, the hopping rate w(ΔE) is determined by the tendency of stem-cells to aggregate, which may be modelled by a drop in free energy ΔE resulting from stem cells migrating towards each other and forming inter-membrane junctions.

### Example 2

### A Hydrodynamic Description of the Average Cell Densities

Referring to FIG. 2, FIG. 2a (Left) shows a solution for the stationary stem cell density as obtained by numerically solving the hydrodynamic equations associated with the cell fate model (see ‘Hydrodynamics Model’ section above). One sees that, on average, stem cells aggregate into a uniform array of dense clusters (white), within a sea of committed progenitor and post-mitotic cells (black).

FIG. 2a (Right): The rate of stem cell proliferation is shown for the same stationary state, revealing that stem cells within each cluster are quiescent, and with division occurring only on the cluster edges. The slow creation of new stem cells through division compensates for the loss of stem cells through differentiation in the bulk of the cluster, and gives a natural mechanism for maintaining the cluster size.

Empirical measures of basal layer morphology; such as the typical cluster size and the stem cell fraction, are determined by the effective division and migration rates of the different cell compartments (FIG. 2b). Here, the rate of stem-cell differentiation (γ_{SA}) is shown to play an important role in determining the size and separation of stem cell clusters. Solid curves show theoretical predictions made by directly analysing the properties of the hydrodynamic equations (see ‘Hydrodynamics Model’ section above); data points show exact results obtained through numerical solution of the same equations.

Thus, referring to FIG. 2, it can be seen from the hydrodynamics that the system indeed describes the formation of a robust and stable pattern, irrespective of initial conditions, giving access to the relations between the cell kinetics and the stable size and separation of stem cell clusters (see ‘Hydrodynamics Model’ section). Notably, we find from the hydrodynamic analysis that the in vivo observations of stem cell clusters containing approximately 40 cells each are consistent with stem cells dividing symmetrically up to 7 times on average before undergoing differentiation into committed progenitors on the cluster boundaries, whereas stem cells within the cluster differentiate at the same slow rate but are incapable of cell division. Moreover, it may be inferred that treatments or conditions leading to an increase in the size of cohesive stem cell clusters will be associated with an increase in the rate of stem cell division relative to differentiation.

### Example 3

### Organotypic Epidermal Cultures

We demonstrate a method for assessing the effect of a treatment on behaviour in a population of cells.

Firstly, a first and a second population of cells are provided. Organotypic cultures, such as cultures prepared on collagen or fibrin rafts seeded with feeder cells, are established using standard methods¹⁻⁵. Two identical cultures comprise the first and second populations of cells in this example.

The treatment is then applied to said first population of cells. In this example, the first culture of normal epidermal keratinocytes is treated with one or more pharmacological agents. The second culture is treated with vehicle only (ie. carrier or solvent of the test agent) as a control. In this example the conventional ‘control’ (ie. the second population) is used to generate the reference value.

Alternatively cultures may be stably transduced with viral or other vectors encoding expression constructs or silencing RNAs with appropriate control vectors⁶⁻⁸.

The first and second populations of cells are then incubated to allow the agent(s) to have any effect(s).

Any altered behaviour in said first population of cells is then detected.

In this example, the effects on patterning in the cultures are analysed by separating the cultured epidermis from the underlying raft and immunostaining for stem cell markers such as β1 integrin, MPSG or LRG1 and/or proliferation markers such as Ki67 or CDC6⁹⁻¹⁵. Patterning is then typically analysed by confocal microscopy of wholemount cultures or analysis of sections of cultures with conventional microscopy^{11,12}. Images are then processed to yield quantitative information on patterning, using standard techniques such as fast fourier transformation.

These images yield information on stem cell differentiation rates, stem cell aggregation, and “partial” information on the division rates of stem cells and committed progenitor cells and the migration rate of post mitotic, terminally differentiated basal cells out of the basal layer.

In this example the reference value is the value determined for said second, population of cells (treated with vehicle only), and detection of altered behaviour in said first population of cells indicates that the treatment has an effect on behaviour in said population of cells.

Therefore, by comparing the output for the first and second populations of cells, it is thus determined if any altered behaviour was caused by the treatment applied.

### Example 4

### Clonal Analysis within Organotypic Cultures

To determine the division rates of stem cells and/or committed progenitor cells and/or the migration rate of stem cells, a variation on the experimental design of example 3 may advantageously be performed.

Cultures are established with constitutively labelled or conditionally labelled keratinocytes in a background of unlabelled keratinocytes. Typically 1 in 400 cells are labelled.

Constitutive labelling may be achieved by the use of stable dyes, such as PKH26 or genetic labels such as a fluorescent protein expressed from a lentiviral or retroviral vector¹⁶⁻¹⁸. Conditional labelling may be achieved by establishing cultures using an inducible retroviral vector, such as a vector in which label expression is induced by cre recombinase, delivered by a second lentiviral or adenoviral vector expressing cre once the culture is established^{19,20}.

A set cultures is established and treated as above: typically 3 cultures are taken for analysis at a series of time points, such as 1, 2, 4, 8 16 and 32 days, and immunostained for stem cell and/or proliferation markers. In this example the first and second populations of cells are the treated and control cells at each timepoint.

The number of cells in each of approx. 50-200 clones per culture is scored using confocal microscopy²¹.

The behaviour of clones inside and outside stem cell clusters is analysed to determine the stem cell and committed progenitor cell division rates and the rate of migration of post mitotic basal cells into the basal layer, as well as stem cell differentiation rates and stem cell aggregation capacity. These clonal analyses are advantageously performed to further characterise phenotypes identified in example 3.

### Example 5

### Analysis of Pattern Formation from Single Cell Suspensions

Information on stem cell clustering may also be obtained by following pattern formation following the plating single cell suspensions of keratinocytes.

In this example a population of keratinocytes labelled with a marker such as a fluorescent dye or a genetic label such as a retrovirally expressed fluorescent protein is allowed to attach to an extracellular matrix protein such as type IV collagen for 10-20 minutes, during which time stem cells will attach^{10,11}.

The non adherent cells are removed and unlabelled keratinocytes which have been panned on the same matrix protein to remove stem cells are plated on top of the adherent labelled cells.

The cells used in this example are wild type keratinocytes treated with a control (eg. vehicle or carrier solvent) or an experimental agent (eg. a candidate drug or pharmacological agent) for the second and first populations of cells respectively.

Cluster formation as indicated by aggregation of the labelled cells typically occurs over the next 15-20 hours. Thus the cells are incubated for 15-20 hours.

Analysis of pattern formation in the resultant sheets of keratinocytes at 24 hours yields information on stem cell aggregation. This may be advantageously used to give a rapid indication of agents that merit further investigation as in previous examples.

Time lapse microscopy may also be used to track the cluster formation.

### Example 6

### Pattern Formation in Single Cell Clones

A further approach is the analysis of pattern formation in large clones (also known as holoclones) derived from single cells²².

As above, the cells used may be wild type keratinocytes treated with a control or an experimental agent, cells transfected with experimental or control siRNA or cells expressing a control or experimental gene or silencing cassette.

Patterning of stem cells, committed progenitors and differentiated basal cells as revealed by immunostaining appears in clones containing as few as 150 cells that develop within 5 days of culture.

Analysis of larger colonies at 10-14 days of culture reveals well developed patterning in the centre of the colony in control cultures whilst the proportion of stem cells is enriched at the periphery of the colony¹¹.

Analysis of patterning in single cell clones may be advantageously used to determine the stem cell and committed progenitor cell division rates and the rate of migration of post mitotic basal cells into the basal layer, as well as stem cell differentiation rates and stem cell aggregation capacity.

### Example 7

### Detailed Biological Readouts

Clearly the invention may be applied in many ways now that the model of epithelial homeostasis has been set out. This example illustrates a number of ways in which detailed insights into biological events may be obtained according to the present invention.

In the following pages, guidance is given regarding what to examine or determine, and what inferences or information is thereby extracted based on the model herein.

In particular, the columns marked ‘Experimental Observables’ relate to characteristics of the population which may be determined.

The columns marked ‘Response in Control Sample’ relate to reference values—in accordance with the present invention these are determined either by experiment or by prediction (or description) by the hydrodynamic model. These reference values may advantageously be directly used if desired by the operator.

The columns marked ‘Novel changes to look for during experiment’ represent teachings how the characteristics may advantageously be determined or interrogated.

The columns marked ‘Cell kinetics potentially affected’ provide guidance regarding relating the model to the characteristics of the cell populations. This is particularly relevant to the detailed use of the model to analyse or investigate cell behaviours (third-level or detailed level of analyses).

The flow charts presented in the figures embody and describe further methods which may form part of the invention. Subsets from the complete charts of method steps presented may be independently formulated as methods. For example, with reference to FIG. 3A, any one of or any combination of the four causes presented in the flow chart might be investigated to form a method—the method does not necessarily require each step presented in the flow chart(s) to be performed, only the steps required to produce a meaningful analysis (which may include elimination of a cause ie. a negative result rather than strictly requiring absolute determination of the effect).

With reference to the table entitled ‘Application of model reveals changes in cell kinetics’, the small ticks represent ‘partial’ information. In fact this information is robust and useful, the designation of ‘partial’ information merely indicates that the readout is narrowed to one of a small number of options or variables, for example the precise individual variable may not have been determined in that embodiment. If it is desired to pin down the precise variable which has been altered, this can be performed according to the guidance set out above.

### Example 7 Continued

### Further Applications and Embodiments

We relate a novel model of epidermal maintenance in mammals such as humans to the analysis of new environmental conditions, including drug application and genetic mutation.

Several cell kinetic parameters (see below) characterise the behaviour of keratinocytes.

These parameters are accessible through a range of experiments (see below), and primarily from their signature on the spatial structure of confluent keratinocyte sheets in culture.

For each experiment, we summarise the changes in empirical results (compared to a control experiment) that are indicative of respective changes to cell kinetics.

| Cell kinetic parameters | Proposed experiments |
|---|---|
| Stem-cell division rate | Organotypic culture (A) - morphology |
| Stem-cell differentiation rate | Organotypic culture (B) - clonal |
| (Stem->CPC) | analysis |
| Stem-cell adhesion capacity | Holoclone (A) - morphology |
| CPC division rate | Single-cell seeding (B) - clonal |
| | analysis |
| DC (differentiated or post- | Rapid aggregation from homogenous |
| mitotic) migration rate | plating |

### Expt. (1/5): Organotypic Culture (A)—Morphology

### Observables

### Experiment Description

Apply treatment to, ready-made Organotypic keratinocytes culture, leading to steady-state after several weeks.

Basal layer stained for stem, proliferation and differentiation markers.

| Experimental | Response in control | Novel changes to look for during |
|---|---|---|
| observables | sample^{A} (Overview) | experiment |
| S-cell fraction | 25-40% of basal layer | Change in quiescent S-cell |
| | | fraction |
| | | Activity in S-cell compartment |
| | | Change in CPC/DC ratio |
| Fraction and | Excluded from S-cell | |
| location of | clusters | |
| proliferating | 15-25% of non-S-cell | |
| cells (e.g ki67- | compartment (TBC) | |
| stained) | | |
| S-cell cluster | Cohesive clusters | Changes to cluster morphology |
| morphology | 6-9 cells diameter | Cluster avg. size grows/shrinks |
| | | Clusters become stripe-like |
| | | Clusters vanish through S-cell |
| | | disaggregation |
| Fraction | Excluded from S-cell | Change in CPC/DC ratio |
| differentiated | clusters | |
| cells | 75-85% of non S-cell | |
| | compartment (TBC) | |

^{A}Control - Normal keratinocytes in absence of drug/mutation/environment changes outside protocol.

### Expt. (1/5): Organotypic Culture (A)—Morphology

Cell Kinetics Revealed from Experiment See FIG. 3

| | Novel changes to look for | |
|---|---|---|
| Experimental observables | during experiment | Cell kinetics potentially affected |
| S-cell franction | Change in quiescent S-cell | “δ” ratio^{A} modified |
| Distribution of proliferating | fraction | S-cell density regulation disrupted |
| cells | Activity in S-cell | Ratio of DC-migration rate to CPC- |
| | compartment | division rate changed |
| | Change in CPC/DC ratio | S-cell division/differentiation rates |
| | Changes to cluster | changed |
| | morphology | |
| | Cluster avg. size | |
| | grows/shrinks | |
| S-cell cluster morphology | Clusters become stripe- | Feature of growing S-cell fraction, |
| | like | see above |
| | Clusters vanish through | S-cells activated (proliferating), or |
| | S-cell disaggregation | S-cell adhesion disrupted |

^{A}δ = (γ_{BB} − γ_{AA})γ_{BO}/[(2γ_{BB} + γ_{AB})γ_{SA}]

### Expt. (2/5): Organotypic Culture (B)—Clone Analysis

### Observables

### Experiment Description

Apply treatment to ready-made organotypic keratinocyte culture, leading to steady-state after several weeks.

Use genetic (or other) labelling to track representative sample of basal layer cells and their progeny over a period of weeks to months

| Experimental | Response in control sample | Novel changes to look for |
|---|---|---|
| observables | (Overview) | during experiment |
| Clone size | Early-stage: clone size | Changes to clone size |
| distributions | follows CPC-cell Galton- | distributions and morphology |
| | Watson statistics | at early, medium and late |
| | Mid-stage (weeks-months): | times |
| | CPC/DP clone population | |
| | distribution becomes quasi- | |
| | stationary | |
| | Late-stage (months): Clone | |
| | size follows stem-cell | |
| | Galton-Watson statistics | |
| Clone | At early/mid-stage, clones | |
| morphology | irregular | |
| | At late stages, most clusters | |
| | contain discrete stem-cell | |
| | clusters | |

### Expt. (2/5): Organotypic Culture (B)—Clone Analysis

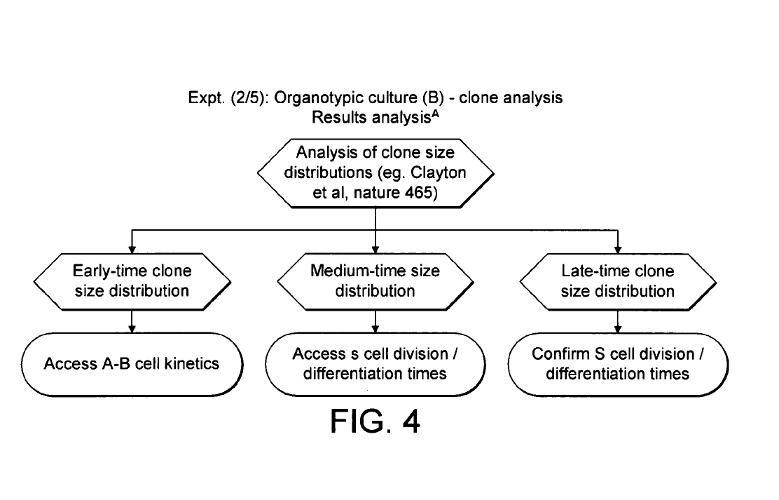

Cell Kinetics Revealed from Experiment See FIG. 4

| | Novel^{B} changes | |
|---|---|---|
| Experimental | to look for | |
| observables | during experiment | Cell kinetics potentially affected |
| Clone size | Changes to clone size | All effective rates of cell division, |
| distributions | distributions and | Differentiation and migration - |
| | morphology at early, | analyse clone size distribution |
| | medium and late times | |
| Clone | Change in clone | To be determined |
| morphology | cohesiveness | |
| | Change in clone | |
| | fraction in | |
| | stem cell compartment | |

### Expt. (3/5): Holoclone (A)—Morphology

### Observables

### Experiment Description

Track holoclone growth from single cell under novel environmental/genetic/drug influence

Study holoclone growth rate and morphology, including spatial distribution of S, A, B cells.

| | Response in control sample^{A} | Novel changes to look for during |
|---|---|---|
| Experimental observables | (Overview) | experiment |
| Holoclone shape | Cohesive, obtuse | Holoclone decoherence and shape |
| | Actige region on holoclone | irregularity |
| | edge measures 5-15 cells | Change in radial holoclone profile |
| | thickness | (e.g. edge thickness) |
| S-cell pattern and | Holoclone center mimics | For holoclone center, all change |
| proliferating cell | structure of normal | indicators as for “organotypic |
| distribution | epidermis (both in stem cell | culture (A)”, (expt. (2/5) |
| | morphology and | Change in stem cell edge fraction |
| | proliferation activity) | Change in edge activity |
| | Holoclone edge contains | |
| | high stem cell fraction and | |
| | increased proliferation | |
| | activity | |

^{A}control - Normal keratinocytes in absence of rug/mutation/treatment/environmental changes

### Expt. (3/5): Holoclone (A)—Morphology

Cell Kinetics Revealed from Experiment See FIG. 5

| | Novel changes to look for | |
|---|---|---|
| Experimental observables | during experiment | Cell kinetics potentially affected |
| Holoclone shape | Holoclone decoherence and | Stem cell cohesiveness, A-cell |
| | shape irregularity | fraction |
| | Change in radial holoclone | Stem cell division/differentiation |
| | profile (e.g. edge thickness) | rates |
| S-cell pattern and | For holoclone center, all | See “organotypic culture (A)”, |
| proliferating cell | change indicators as for | (expt. 2/5) |
| distribution | “organotypic culture (A)”, | Stem cell division/differentiation |
| | (expt. 2/5) | rates |
| | Change in stem cell edge | |
| | fraction | |
| | Change in edge activity | |

### Expt. (4/5): Holoclone (B)—Clonal Analysis

### Observables

### Experiment Description

Grow holoclone from single cell seeding experiment under novel environmental/genetic/drug influence.

At early/mid-growth (equivalent of days—1 week in control), label samples of cells within holoclone and track size, morphology and cell type of clonal progeny.

| | | Novel changes |
|---|---|---|
| | Response in control sample | to look for during |
| Experimental observables | (Overview) | experiment |
| Clone size distributions | | Clonal analysis |
| and morphology | | |
| At holoclone center | At center, clonal evolution | |
| | mimics organotypic culture | |
| | (see expt. 3/5) | |
| At holoclone edges | At edges, average clone | |
| | size grows faster than | |
| | holoclone growth rate | |
| | “Edge” clone shape | |
| | irregular | |

Control—Normal keratinocytes in absence of drug/mutation/environmental changes outside protocol

### Expt. (4/5): Holoclone (B)—Clonal Analysis

Cell Kinetics Revealed from Experiment

| | Novel changes | |
|---|---|---|
| Experimental | to look for | Cell kinetics |
| observables | during experiment | potentially affected |
| Clone size distributions | Clonal analysis | All cell kinetics potentially |
| and morphology | | accessible from clone size |
| At holoclone center | | distributions at center of |
| At holoclone edges | clone | |

### Experiment (5/5): Rapid Aggregation

### Observables

### Experiment Description

Representative keratinocye samples in homogenous suspension plated in uniform culture

Samples stained for stem and proliferation markers at intervals up to (e.g.) 48 hours

| | Response in control sample | Novel changes to look for during |
|---|---|---|
| Experimental observables | (Overview) | experiment |
| Formation of confluent | Partial at 8 hours | Inhibited/accelerated time to |
| layer | Full at 18-24 hours | confluence |
| Number and location of | High at 8 hours | Shorter/prolonged proliferation |
| cell stained for | Drops at confluence (24 | period |
| proliferation marker (e.g. | hours) (Allon: TBC) | |
| ki67) | | |
| Stem cell spatial | Cohesive clusters visible | Cluster morphology changes |
| distribution (degree of S□- | (at 8 and 24 hours) | Cluster larger/smaller |
| cell aggregation) | Average of 10-20 cells per | Irregular shapes |
| | cluster (Allon: TBC) | |

Control—Normal keratinocytes in absence of drug/mutation/environmental changes outside protocol

### Experiment (5/5): Rapid Aggregation

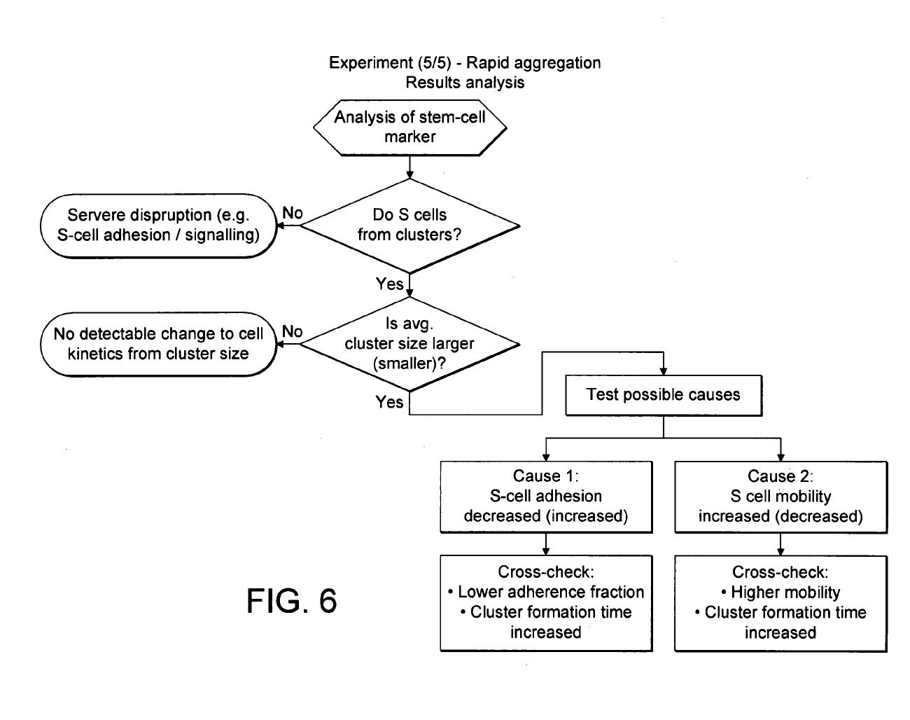

Cell Kinetics Revealed from Experiment See FIG. 6

| | Novel changes to look for | |
|---|---|---|
| Experimental observables | during experiment | Cell kinetics potentially affected |
| Formation of confluent | Inhibited/accelerated time | Cell motility decreased/increased |
| layer | to confluence | Cell-cell signalling and junction |
| | | formation changed |
| | | UPSD (UPSD - Unknown |
| | | Potentially Serious Distruption) (in |
| | | case of no confluence) |
| Distribution of proliferating | Shorter/prolonged | Correlation with time-to- |
| cells | proliferation period | confluence (normal density |
| | | regulation) |
| | | Density regulation of proliferation |
| S-cell aggregation | Cluster morphology | Stem cell adhesion changed |
| | changes | Stem cell motility |
| | Cluster larger/smaller | |
| | Irregular shapes | |

### Application of Model Reveals Changes in Cell Kinetics

### Accessible Cell Kinetic Rates

| | | | Stem-cell | | Stem-cell |
|---|---|---|---|---|---|
| | CPC division | DC migration | division | Stem-cell | adhesion |
| Experiment | rate | rate | rate | diff. rate (S −> A) | capacity |
| Rapid aggregation | | | | | ✓ |
| from homogenous | | | | | |
| plating | | | | | |
| Organotypic culture | ✓ | ✓ | ✓ | ✓ | ✓ |
| (A) − morphology | | | | | |
| Organotypic culture | ✓ | ✓ | ✓ | ✓ | ✓ |
| (B) − clonal | | | | | |
| analysis | | | | | |
| Holoclone (A) − | | | ✓ | ✓ | ✓ |
| morphology | | | | | |
| Single-cell seeding | ✓ | ✓ | | | ✓ |
| (B) − clonal | | | | | |
| analysis | | | | | |

### Example 8

In the basal layer of human interfollicular epidermis, stem cells aggregate into near-quiescent clusters, separated by proliferating and differentiating keratinocytes. Remarkably, this pattern is reconstituted in vitro. Combining a wide range of existing observations with new experimental data, we elucidate the origin of patterning and quiescence in homeostatic tissue, and explain the ability of stem cells to restore patterning in culture and thereby reconstitute their niche. This behaviour points at a simple set of organisational principles controlling stem and progenitor cell fate, and provides a unified model of epidermal maintenance in mouse and human. In particular, we show that epidermis is maintained by a committed progenitor cell population whose stochastic behaviour enables stem cells to remain largely quiescent unless called upon for tissue repair.

### Introduction

By drawing on a wide range of experimental data, we show that epidermal stem and progenitor cell behaviour conforms to a simple set of organisational principles that explains not only the clustering of quiescent stem cells in normal tissue, but also their ability to recreate this niche in vitro.

Although the general architecture of human epidermis parallels that of mouse, there is strong experimental evidence for proliferative heterogeneity within the basal layer cell population in human. A pioneering study demonstrated that sub-cloning single cell-derived colonies of cultured human keratinocytes defines three types of colony (Barrandon and Green, 1987b): those with a very high proliferative potential that give rise to large circular colonies when subcloned (termed holoclones); those with very limited proliferative potential that give rise to small irregularly shaped colonies (paraclones); and colonies with intermediate properties (meroclones). Subsequent studies showed that cultured keratinocytes could be fractionated on the basis of their expression of the β1 integrin family of extracellular matrix receptors (Jones and Watt, 1993). Cells expressing high levels of β1 integrin form large actively growing colonies and regenerate human epidermis when grafted onto immunocompromised mice consistent with stem cell behaviour. In contrast, those expressing lower levels form small abortive colonies in which all cells undergo terminal differentiation and are unable to regenerate epidermis in xenografts (Jones et al., 1995).

Analysis of integrin expression in human epidermis reveals that the basal layer is organised into irregular clusters of keratinocytes expressing high levels of β1 integrin, which appear to be localised around the tips of dermal papilla (Jones et al., 1995). Interspersed between the clusters are regions of lower integrin expression. Moreover, cells expressing high levels of other stem cell markers (the notch ligand Delta and the cell surface proteins MCSP and LRIG1) are also clustered, and co-localise with β1 integrin^{high} cells (FIG. 8A) (Estrach et al., 2007; Jensen and Watt, 2006; Legg et al., 2003; Lowell and Watt, 2001). In contrast, the desmosomal protein, desmoglein3, has a reciprocal distribution, localising in regions of β1 integrin^{Low} expression (Wan et al., 2003). A striking feature of the clusters is that the great majority of the constituent cells are quiescent (FIG. 8A). Proliferating and post-mitotic basal layer cells lie between the clusters (Jensen et al., 1999). Taken together, these results suggest that, in human IFE, stem cells aggregate into cohesive clusters of near-quiescent cells, interspersed with cycling and differentiating cells with a much lower proliferative potential (Jensen et al., 1999; Jones et al., 1995).

Superficially, the evidence for stem cell clustering within a reciprocal pattern of proliferating and differentiating cells in human IFE is in marked contrast to murine epidermis where no clustering is apparent and proliferating and differentiating keratinocytes appear to be scattered randomly throughout the IFE (Braun et al., 2003). Whether stem cell patterning in human WE is capable of revealing new aspects of stem cell behaviour depends, in part, on whether stem cells are themselves responsible for clustering, or whether the pattern results from some external process that is not influenced by stem cell behaviour. Although the role of external factors cannot be ruled out, it is significant that, when placed in culture, keratinocytes spontaneously reconstitute the in vivo pattern (FIG. 8B) (Jones et al., 1995).

Historically, the experimental results from the studies of human epidermis reviewed above have been interpreted within the framework of the classical stem/TA model. Cells residing between stem cell clusters have been thought to represent the short-lived TA cell population that is continuously replenished by the stem cell compartment. However, this interpretation has been challenged recently by a powerful experiment in which human skin was grafted onto immunocompromised mice and then transduced with lentiviral reporter vectors. When the epidermis was examined six months later, the persisting clones were found to have a wide range of size and shape, and appeared to originate not only from basal cells within β1 integrin^{High} clusters, but also from cells between clusters (Ghazizadeh and Taichman, 2005). If one assumes that long-lived clones can only arise from the stem cell population, a tenet of the classical stem/TA cell hypothesis, these findings appear to conflict with the evidence for stem cell clustering. However, if human epidermis has a population of non-stem progenitor cells with the potential to undergo an unlimited number of cell divisions prior to differentiation, as seen in murine IFE (Clayton et al., 2007), long-lived clones could arise from both stem cell clusters and the intervening cells.

### Model of Epidermal Maintenance

Although the classical stem/TA cell model has been used widely to interpret experimental data, it does not attempt to engage with the spatial organisation (clustering) and regulation (quiescence) of stem cells in human tissue. Yet, the regeneration of stem cell patterning and quiescence in culture (FIG. 8B), which can occur even without external signals from other cell types such as dermal fibroblasts, is suggestive of a general organisational principle involving the cooperative behaviour of the keratinocyte population. Drawing upon new clonal fate data, we demonstrate that the observed patterning behaviour is consistent with a simple model of epidermal maintenance, involving a stem and stochastic CP cell population, that:
elucidates the relationship between stem cell patterning and quiescence in homeostasis;
explains the ability of stem cells to reconstitute patterning and quiescence in culture, thereby regenerating their niche;
and reveals how the maintenance of human and murine epidermal homeostasis can be embraced within a single framework.

To develop this model, we will begin with a quantitative analysis of clonal fate data obtained from a study of primary human keratinocytes. As well as reinforcing the existing evidence for two progenitor cell populations, this investigation demonstrates that human non-stem progenitor cells behave as a stochastic CP cell population. Then, drawing on qualitative observations of the stem cell behaviour in vivo and in vitro, we propose a two-compartment model of IFE—a “stem/CP cell” model. Starting with a simple qualitative explanation for the observed patterning behaviour in human IFE, in the following section we will develop a “coarse-grained” hydrodynamic theory of basal layer cell fate. Although the stem/CP cell model, and the associated hydrodynamics, does not seek to address the complex molecular circuitry that is responsible for the regulation of cell division, differentiation, and migration, they offer new insights into stem cell regulation and permit robust predictions regarding cell behaviours.

### Clonal Analysis of Human Epidermal Cultures and the Stem/CP Cell Model

As discussed above, early studies proposed a functional definition of stem and non-stem cell progenitor cell populations according to their colony-forming efficiency (Barrandon and Green, 1987b; Jones and Watt, 1993). However, no attempt has been made to combine progenitor cell fate data at single cell resolution with quantitative modelling. By contrast, clonal analysis of mouse epidermis has allowed the properties of a CP cell population to be discerned (Clayton et al., 2007). To what extent can clonal analysis provide insight into progenitor cell fate in human epidermis? To address this question, we performed a quantitative analysis of primary human keratinocytes cultured at clonal density (Barrandon and Green, 1987b; Jones et al., 1995). Cultures were fixed at 24 hours to 7 days after plating, stained for keratin 14 or E cadherin to detect keratinocytes, and Ki67 to identify proliferating cells, after which clone size was scored by fluorescence microscopy (FIG. 9A).

Previous studies indicate that stem cells give rise to macroscopic colonies, whereas the remaining smaller clones derive from progenitors committed to terminal differentiation (Barrandon and Green, 1987b; Jones and Watt, 1993). Indeed, at 7 days post-labelling, the population of clones has a wide size distribution, with almost half of clones containing 16 or fewer cells, whilst approximately 15% of clones contain over 256 cells (FIG. 9A). The very largest clones contain as many as 3,000-4,000 cells (supplementary FIG. 13). In order to systematically identify the cell fate characteristics of stem and non-stem progenitor cell populations, clones were separated on the basis of their size and fraction of proliferating cells (FIGS. 9B, 9C). Large clones and clones that contained over 50% Ki67 positive cells (termed “type I” clones) were considered separately from the remaining, “type II”, colonies (see methods and supplementary section S-I for details). If type I and II clone populations derive from different cell types, then it should follow that proportion of each type of clone remains the same. Indeed, this ratio is roughly constant from 48 hours onwards, with 40±2% of all clones of type II.

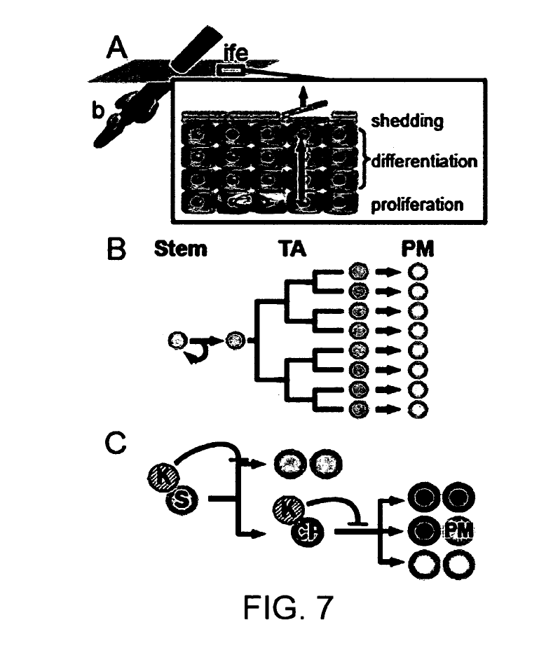

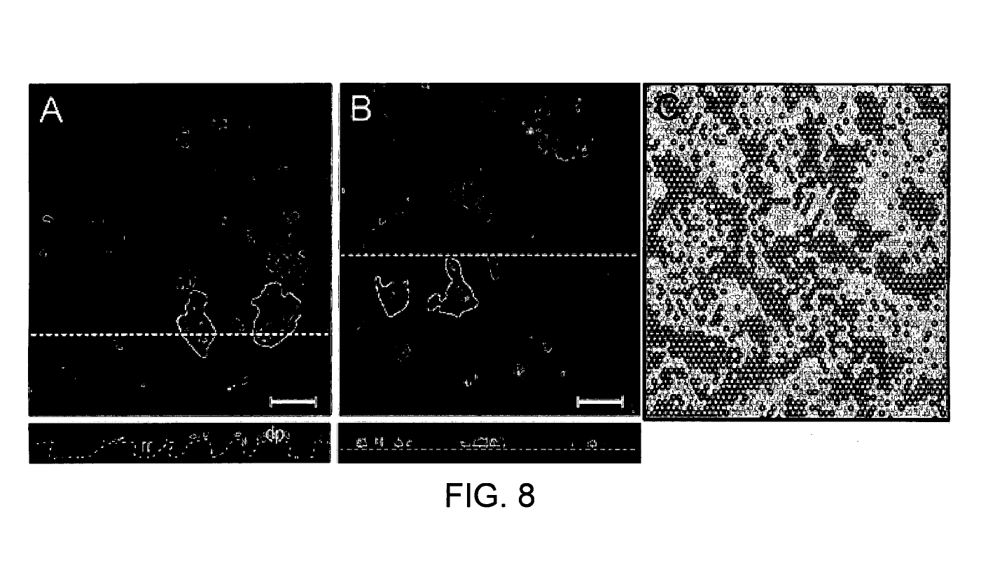

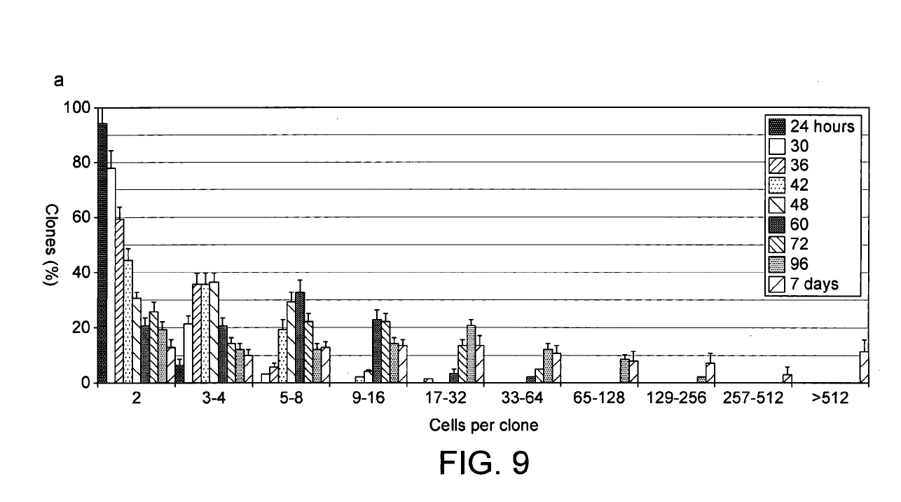

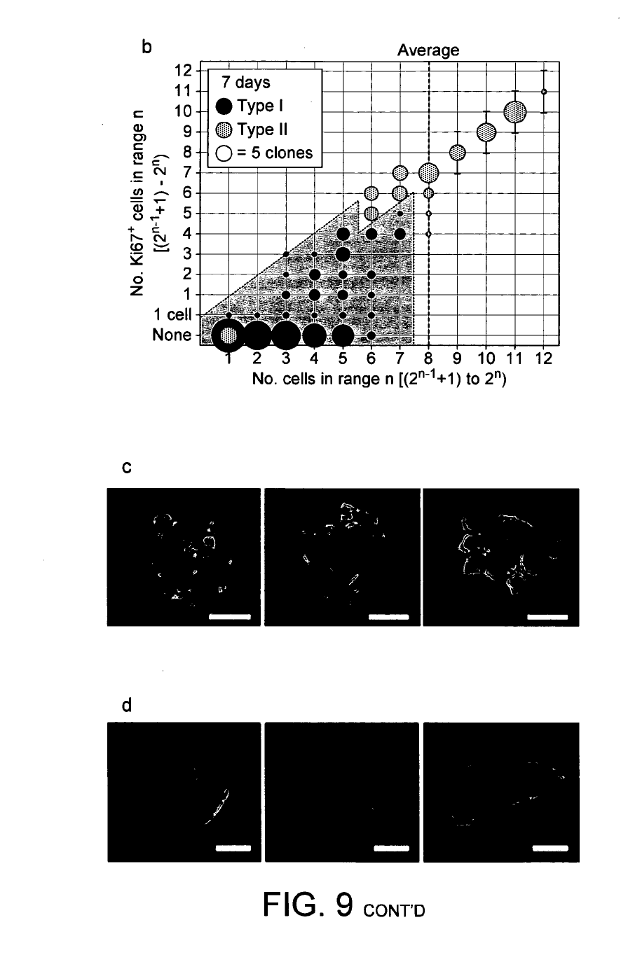

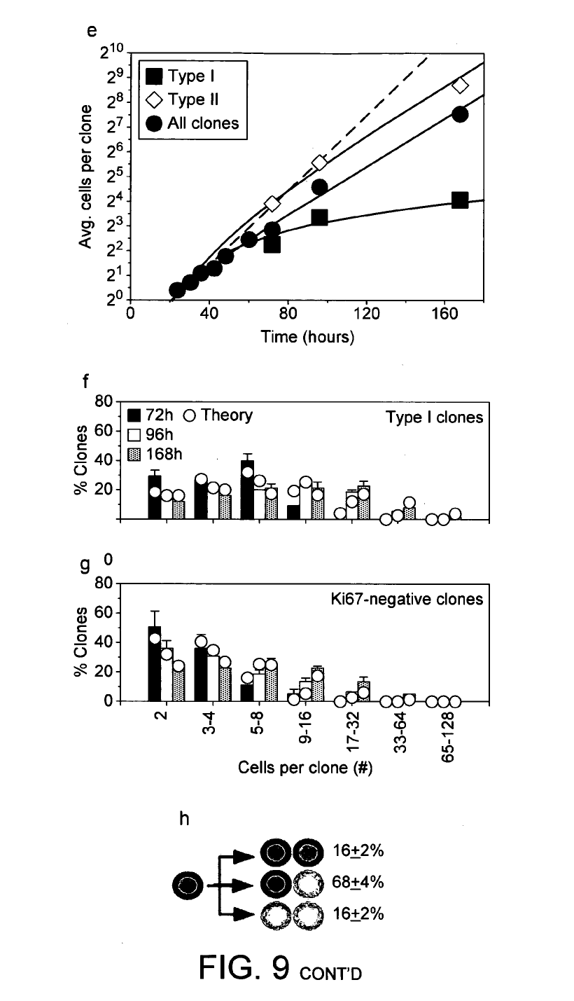

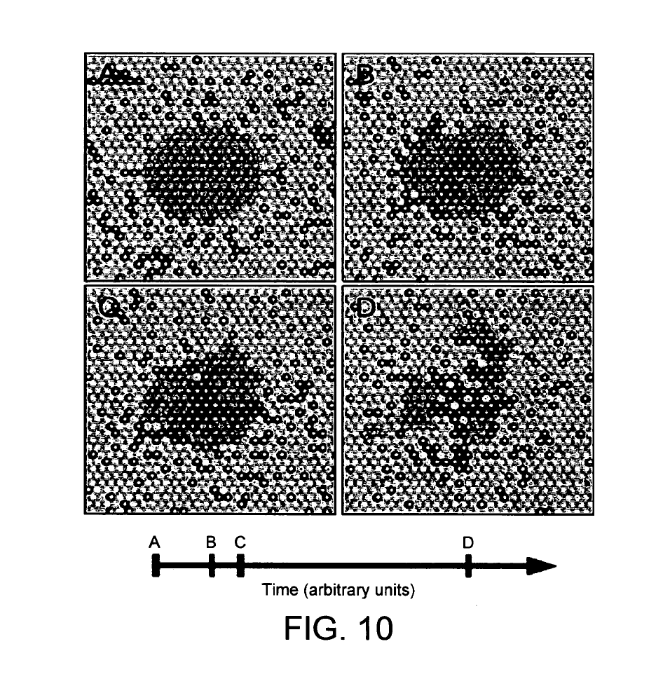

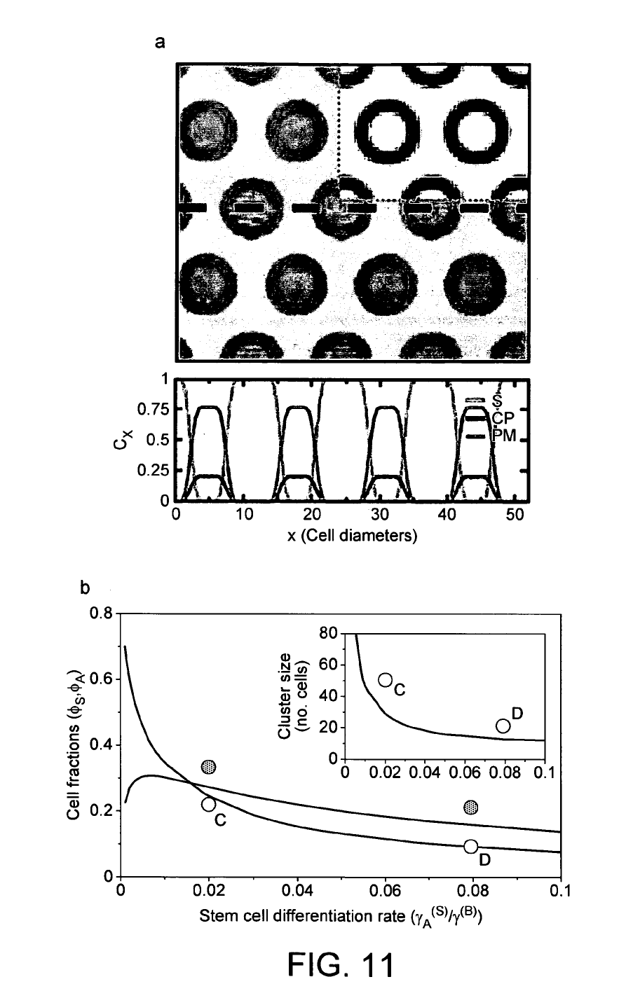

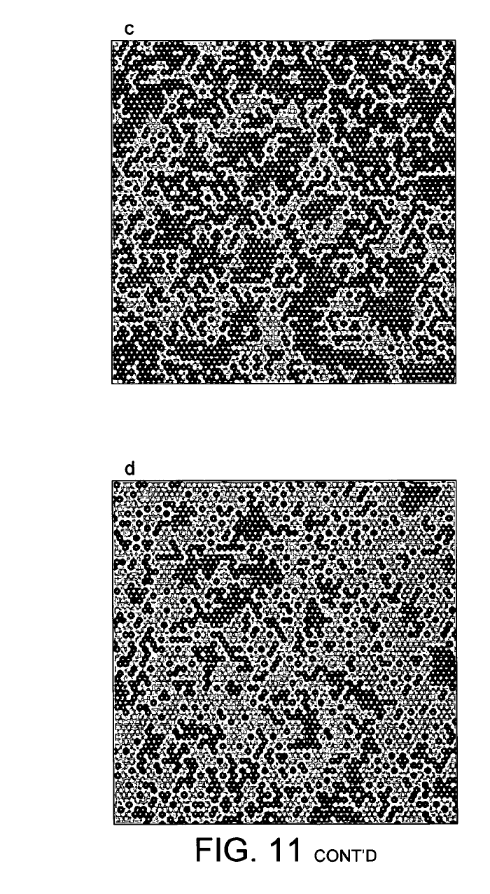

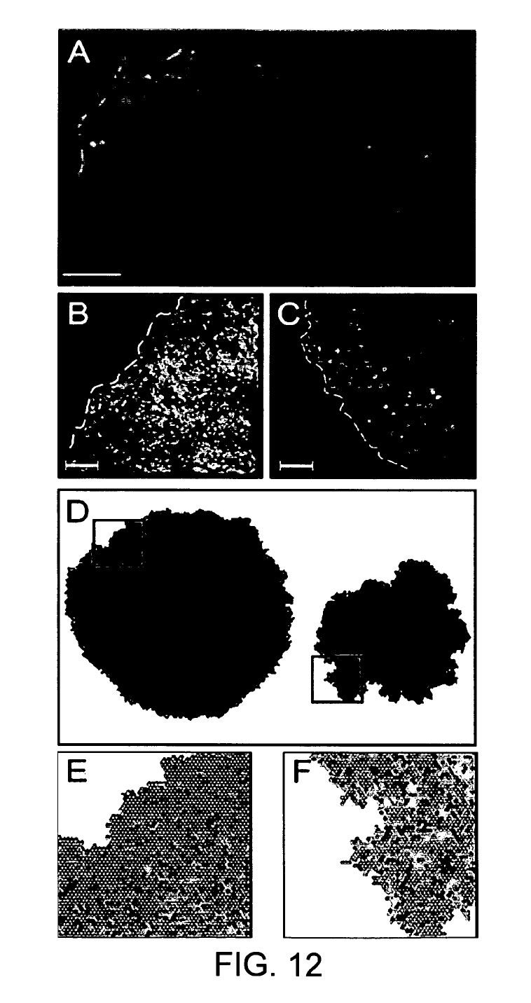

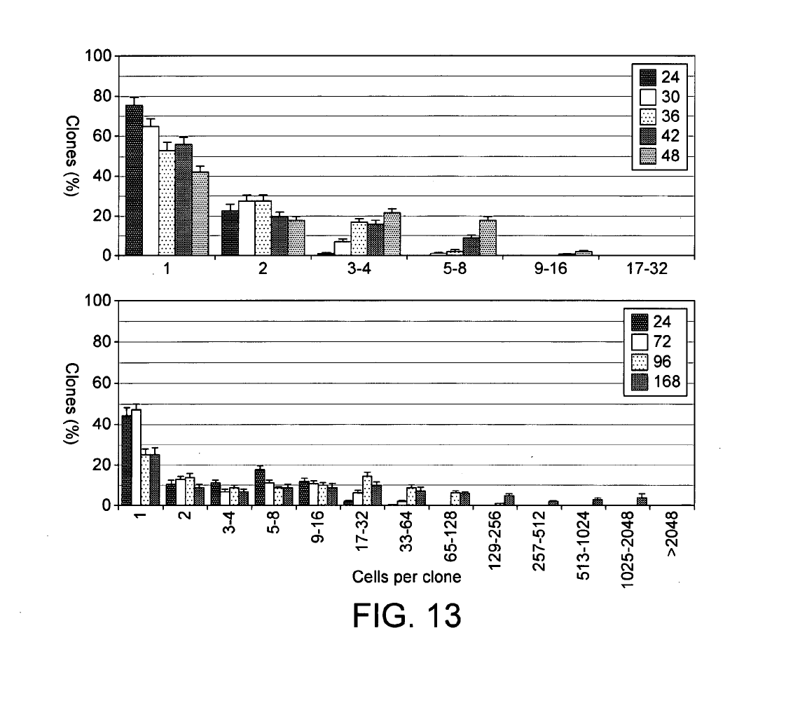

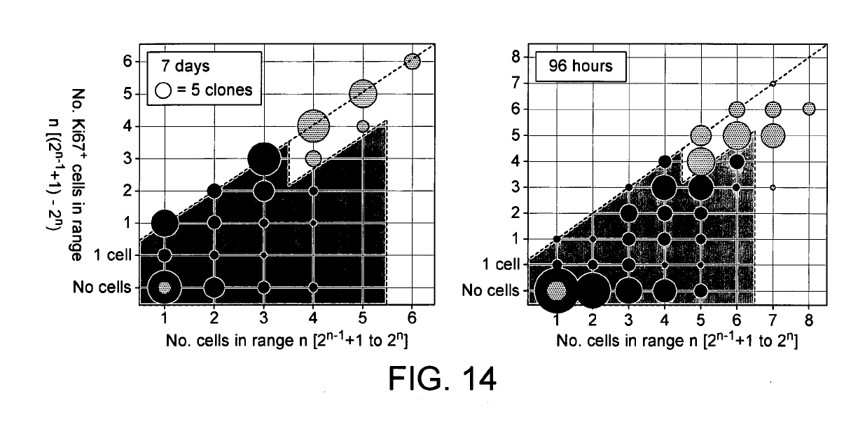

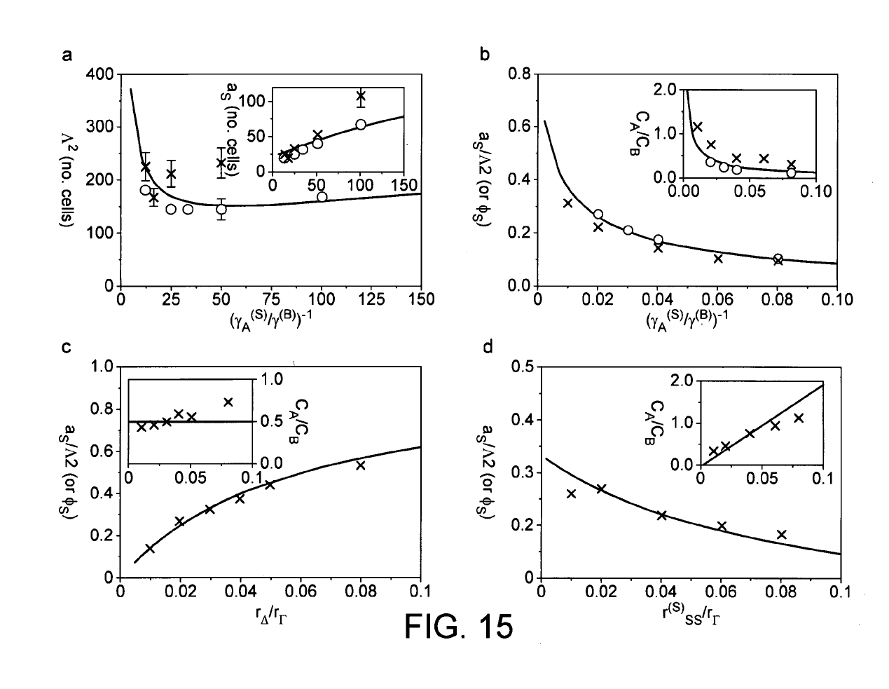

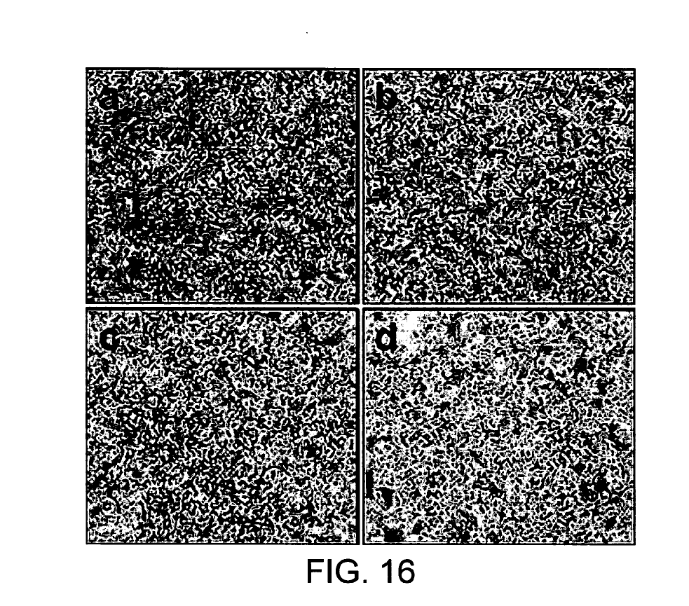

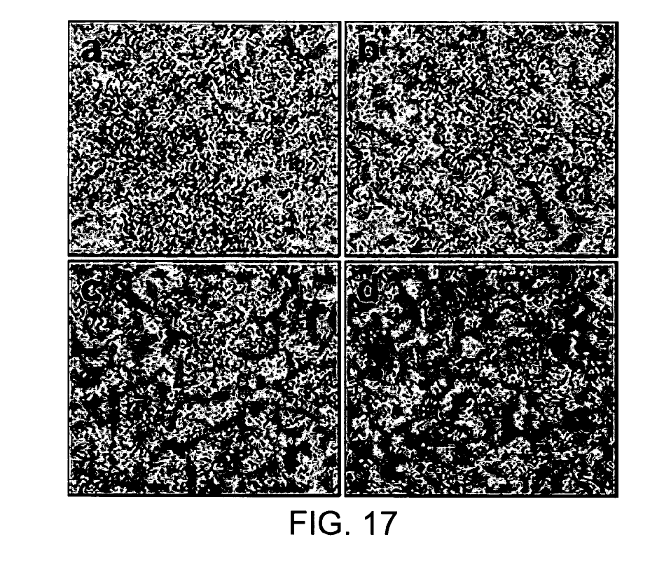

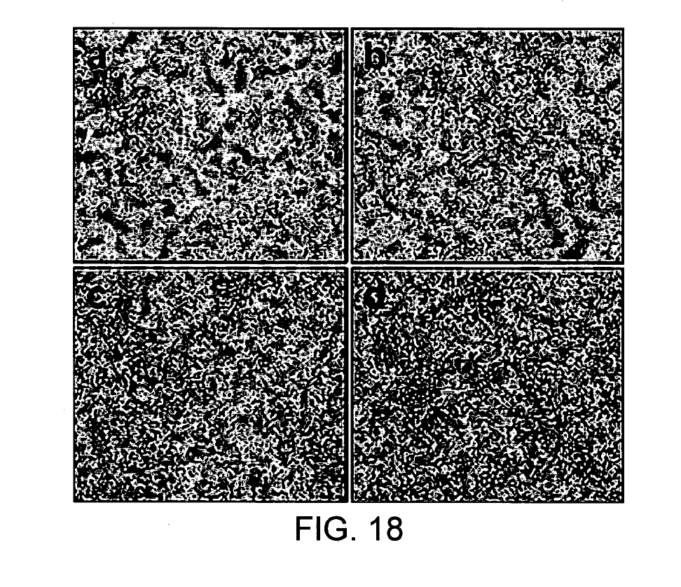

A striking feature of the type I population is that the average size of the clones grows linearly with time (FIG. 9E). Moreover, Ki67 staining of two cell clones reveals that a progenitor cell may give rise to two cycling cells, two non-cycling cells, or one cycling and one non-cycling daughter cell (FIG. 9D). Although such behaviour is difficult to reconcile with a classical TA cell population, both observations are consistent with stochastic CP cell behaviour of the form identified in murine epidermis (FIG. 7C) (Clayton et al., 2007; Klein et al., 2007). Indeed, the linear increase in average clone size is a signature of balanced symmetric fate with CP cell division leading to two cycling or two non-cycling cells with equal probability.

To test whether type I clones derive from CP-type cells, it is instructive to turn to the detailed size distribution. The stochastic fate of CP cells can be characterised simply by the average cell cycle time, and the fraction of cells undergoing symmetric versus asymmetric division. The observed linear increase in average type I clone size (FIG. 9E) translates to an average cell cycle time of 10±2 hours in vitro (r²=0.97, standard error from regression analysis; see supplementary section S-I). Taking this value of the cell cycle time, application of the balance CP cell model provides a single parameter fit to the entire clone size distribution, with the asymmetric division probability lying in the range 64-72% (FIGS. 9F, 9H). Significantly, the same parameters accurately predict two independent experimental datasets; the distribution of the number of cycling cells per clone over time (supplementary FIG. S4), as well as the size distribution of fully differentiated clones (FIG. 9G).

Clonal culture conditions provide a highly artificial environment. It is, therefore, remarkable that non-stem progenitor cell behaviour coincides with that observed in vivo in mouse. This suggests that such stochastic CP cell behaviour is largely insensitive to extrinsic regulation and, therefore, is likely to characterise the cell behaviour in vivo in human WE. Although it is difficult to test this assertion directly, the observation of long-lived clones with a wide range of sizes arising from cells outside stem cell clusters in human epidermal xenografts (Ghazizadeh and Taichman, 2005) is typical of cells capable of an unlimited number of rounds of division before terminal differentiation, a hallmark of the CP cell population (Clayton et al., 2007; Jones et al., 2007; Klein et al., 2007). We are therefore led to conclude that both in vivo and in vitro, human non-stem progenitor cells behave in a similar stochastic manner as CP cells in murine tail skin (FIG. 9H).

Turning now to type II clones, their average size increases exponentially with time (FIG. 9E). We shall identify these clones as deriving from self-renewing stem cells. Intriguingly, Ki67 staining reveals that a proportion of cells within type II clones are non-cycling (FIG. 9B and supplementary FIG. 14), indicating that stem cells differentiate even at the earliest stages of clonal growth in vitro. The significance of stem cell differentiation is further signalled by a departure of the average type II clone size data from a simple exponential curve (FIG. 9E, dashed line). By contrast, one can find a fit to the average clone size if one-assumes an average stem cell cycle time of 13 hours, while allowing for stem cell differentiation into CP cells at a rate equivalent to 50% of the rate of cell division (FIG. 9E). This translates to a population doubling time of 21 hours, a figure consistent with previous studies (Barrandon and Green, 1987a). These results reveal that type II cells exhibit a range of potential behaviours that are expected of a stem cell compartment. In the following we will consider a model in which stem cells may undergo symmetric division into two daughter stem cells, or differentiate to form a progenitor cell committed to terminal differentiation (FIG. 7C). Although one could conceive of other channels of stem cell fate, such as asymmetric division, such generalizations will not effect the large-scale organisation of the tissue, the focus of the following discussion.

Although the observations of colony growth at early times in clonal culture are consistent with rapid proliferation of both the stem and CP cell populations, the eventual regeneration of stem cell patterning and quiescence in organotypic culture (FIG. 8B) suggests that stem cell division is subject to strong extrinsic regulation, with division being inhibited when cells are locally confluent. Similarly, the observation that, in homeostatic tissue, the basal layer cell density remains approximately uniform over time suggests that CP cell proliferation is also tightly regulated during normal tissue turnover: On average; for each cell division, one cell must leave the basal layer through upward migration. Together, these observations are suggestive of a “density-dependent regulation” of the division rate with proliferation becoming halted when the local cell density becomes too high. Such regulation can be achieved by a range of biochemical regulatory mechanisms such as cell-cell trans-membrane signalling, gradients of short-range diffusible signalling factors, or mechanical stress-based control (Shraiman, 2005). Although not crucial for the discussion below, we will also make the simplifying assumption that, in adult epidermis, the vast majority of basal cell divisions are in-plane generating two basal cells (Clayton et al., 2007; Koster and Roop, 2005; Smart, 1970).

Finally, in addition to their division and differentiation potential, we must address the observed tendency of stem cells to aggregate into clusters. As mentioned above, stem cells are more adherent to underlying extracellular matrix proteins than other basal cells, restricting their mobility (Jensen et al., 1999; Jones et al., 1995; Jones and Watt, 1993). Stem cells also adhere more tightly to each other than to other basal cells by virtue of expressing factors that promote cohesiveness, such as the Notch receptor Delta and the cell surface proteoglycan MCSP (Estrach et al., 2007; Legg et al., 2003; Lowell et al., 2000). In the following, we will therefore suppose that the adhesiveness of stem cells constrains their motion and promotes clustering.

This completes our definition of the stochastic stem/CP cell model as applied to human IFE (FIG. 7C). In summary, we will consider a model of epidermal turnover involving a stem and committed progenitor cell population. The loss of stem cells due to differentiation is compensated by their density-regulated self-renewal. Cell division of the CP cell population is also regulated by the neighbouring cell density with fate (symmetric vs. asymmetric) chosen stochastically. In seeking to address the origin of stem cell patterning, previous works have introduced alternative stochastic models of cell fate that place emphasis on adhesion and regulation (Savill and Sherratt, 2003). Here we have focussed on the simplest model that is consistent with experiment.

### Stem Cell Patterning in Human IFE

To understand how the rules of stem cell fate and regulation translate to the patterning behaviour observed in vivo and in confluent cell cultures, it is necessary to understand the interplay of stem cell adhesion with the dynamics of cell division, differentiation and migration. If the symmetric division rates of the CP cell population were perfectly balanced, as observed in the murine system, the migration of post-mitotic cells from the basal layer would be wholly compensated by the production of cells through CP cell division. In this case, stem cell division and differentiation must be fully suppressed. The adhesive properties of stem cells would, in turn, lead to their gradual aggregation into quiescent clusters through a process of “spinodal decomposition” (Cahn and Hilliard, 1958). Without stem cell division or differentiation, this process would continue unchecked leading to the formation of ever-larger cell clusters (Savill and Sherratt, 2003).

If, however, CP cell division is even slightly imbalanced towards terminal differentiation, stem cells must both divide and differentiate to maintain the CP cell population. This has the effect of arresting the growth of stem cell rich clusters: While small clusters form through the combined effects of stem cell adhesion and proliferation, when clusters become too large, the accumulation of CP cells through stem cell differentiation within the clusters leads to their fragmentation (FIG. 10). The typical cluster size is set by the balance between the depletion of stem cells within a cluster through differentiation, and their self-renewal through symmetric division. As a result of the mutual adhesion of stem cells, newly created CP cells are expelled from clusters. Combined with the effect of upward migration of post-mitotic cells, this exclusion leads to the separation of neighbouring clusters and large-scale pattern formation. This behaviour is recapitulated in a numerical simulation of the stochastic cell dynamics of FIG. 7C. The results show a steady-state behaviour involving a slowly fluctuating irregular patterned distribution, closely resembling the patterned structures observed in human epidermis (FIG. 8C).

In short, the rules of cell division and differentiation provide a mechanism for stem cell aggregation and quiescence leading to the generation and maintenance of a pattern of clusters that constitute the stem cell niche. This mechanism is robust; with only the quantitative characteristics of the clusters depending on the particular rates of division, differentiation and migration. Patterning provides a means to regulate stem cell division by allowing the majority of stem cells (contained within clusters) to remain quiescent. Epidermal homeostasis is achieved predominantly through the turnover of CP cells with a small contribution arising from the stochastic differentiation and density-regulated self-renewal of the stem cell compartment. Disruption of tissue serves to mobilise the stem cell population until the spatial pattern is restored.

Although the behaviour of the tissue (the spatial organisation of cells and their activity) can be seemingly inferred from simple qualitative arguments, to what extent can the stem/CP cell model provide quantitative insights into stem cell behaviour? For example, how sensitive is the large-scale organisation of the stem and CP cell populations (such as the stem cell cluster size) to changes in stem cell behaviour? What predictions can be made about the function of stem and CP cells when tissue is wounded, or “driven” far from steady-state (as in culture)? To circumvent an analysis of the potentially complex and stochastic behaviour of individual cells, and establish a robust and predictive theory of cell fate, we will develop a “coarse-grained” or “low resolution” description of the cell dynamics involving the local basal layer cell densities, c(r,t), i.e. the number of cells per unit area averaged over an area spanning several cell diameters.

Since the methodology is routine, (Cahn and Hilliard, 1958; Elliott and Garcke, 1997; Giacomin and Lebowitz, 1996), we will simply outline the basis of the theory referring to the supplementary (sections S-II, S-III) for a more detailed discussion. To discriminate between different cell types in the basal cell layer, the local cell density (defined in units of the cross-sectional area of a typical basal cell) may be subdivided into the sum, c(r,t)=c_{S}(r,t)+c_{A}(r,t)+c_{B}(r,t), of stem (S type), c_{S}(r,t), committed progenitor (A type), c_{A}(r,t), and post-mitotic (B type), c_{B}(r,t), cell densities. Changes in the local cell densities can then be expressed as a “continuity” (or “reaction-diffusion”) equation for each of the three cell types, X=S, A or B,

$$\frac{\partial {c}_{X}}{\partial t} = {R}_{X} - ∇ \cdot {J}_{X} \qquad ( 1 )$$

Processes that change the total number of cells of each type appear as “rates”, R_{X}, which incorporate the average rates of cell division, differentiation, and upward migration. At the same time, changes to the local cell densities may also result from the lateral motion of cells within the basal layer. The resulting redistribution of cell densities is associated with a flow of cells, or flux J_{X}. In the steady-state (homeostasis), the local cell densities become stationary (time-independent), ∂c_{X}/∂t=0, implying that the local division and differentiation rates are exactly balanced by the flux. For example, the change in cell density resulting from the upward migration of a terminally differentiated cell is compensated by the division and lateral migration of a nearby progenitor cell (supplementary FIG. S4). Expressions for R_{X} and J_{X} associated with the proposed stem/CP cell model are given in the methods section.

The coupled continuity equations (1) allow us to study the dependence of the steady-state pattern morphology on the cell division and differentiation rates. Referring to FIG. 11A, the stable stationary solutions exhibit a near-uniform total cell density with an array of stem cell-rich clusters embedded within a sea of CP and post-mitotic cells. The regularity of the pattern is due to the “mean-field” character of the theory, which describes the average behaviour of the population without addressing fluctuations in the size, shape or separation between individual clusters (cf. FIG. 8C, where the behaviour of individual cells is modelled). Stem cells on the boundary of clusters divide slowly, while those deep within the clusters remain quiescent. It is interesting to note that the patterning predicted by the stem/CP cell model can be regenerated even when the initial conditions are far from the steady-state (supplementary FIGS. S8, S11).

Although the detailed structure of the steady-state cell densities can be determined only numerically, the general properties of the solution can be inferred from an analytical treatment (see supplementary section S-III). In particular, one may show that the rates of division, differentiation, and migration are related to the fraction of stem, CP, and post-mitotic cells in the basal layer (denoted respectively by φ_{S}, φ_{A} and φ_{B}) as simple ratios (see methods). It is striking that the size and patterning of the stem cell population is sensitive to the slow rate of stem cell differentiation, γ^{(s)}_{A} (FIG. 11B-D). If we define γ^{(s)}_{SS} as the effective rate of stem cell division (on the boundary of a stem cell rich cluster), the stem cell fraction is found to scale as the ratio,

$${Φ}_{S} ∝ {( \frac{{γ}_{SS}^{( S )}}{{γ}_{A}^{( S )}} )}^{2} .$$

Moreover, with σ defined as the stem cell mobility, the average diameter of the stem cell-rich clusters varies over a wide range of parameters as

$${d}_{S} ∝ \sqrt{\frac{σ}{{γ}_{A}^{( S )}}} .$$

Such dependence may be understood as reflecting the typical length scale at which differentiated cells are generated within a cluster faster than they can diffuse out. Clusters in excess of this size will fragment due to the accumulation of differentiated stem cells. Although cell mobility affects the size of clusters, it does not influence the population fractions φ_{X}. As a result, all other properties of the basal layer pattern, such as the overall length-scale of the pattern (L=d_{S}/φ_{S}), must adjust to accommodate mobility-induced variations in the stem cell cluster size. From these results, the size and separation of the stem cell-rich clusters is readily inferred. In particular, an estimate that the stem cell-rich clusters contain an average of ca. 20 cells (FIG. 8A) suggests that stem cells divide symmetrically up to four times on average before undergoing differentiation into committed progenitors on the cluster boundary. Stem cells within a cluster differentiate at the same slow rate, but are inhibited from division. In addition, the stem-to-CP cell ratio of 40:60 identified from clonal culture is consistent with an imbalance between the symmetric channels of CP cell division being small and of the same order as the stem cell differentiation rate (see methods).

The relationship identified between the pattern morphology and the rates of cell division and differentiation suggests a range of possible experiments in homeostatic tissue. In particular, one may characterise the role of individual genes or signalling pathways by studying their effect on the steady-state pattern in organotypic cultures or epidermal xenografts. For example, a decrease in the rate of stem cell division relative to differentiation, such as might occur in aging, is predicted to result in a decrease in the size of cohesive stem cell clusters (see FIG. 11B-D). Similarly, an increase in the rate of CP cell differentiation should result in a corresponding increase in the stem cell fraction.

This concludes the discussion of the coarse-grained hydrodynamics as implied by the stem/CP cell model of epidermal maintenance. These results suggest that pattern formation of quiescent stem cell-rich clusters observed in human epidermis, and its spontaneous recapitulation in primary human keratinocyte cultures, provide a robust signature of the underlying stem cell behaviour and its regulation. Whilst the properties of keratinocytes are sufficient to explain patterning, it is clear that, in vivo, stem cell clusters are found overlying dermal papillae and are excluded from the intervening rete ridges (FIG. 8A) (Jensen et al., 1999; Jones et al., 1995). The colocalisation of clusters with dermal papillae may be taken as evidence that signals from the dermis contribute to patterning. However, evidence from autografts of cultured epidermis in patients with full thickness burns suggests an alternative explanation. Following grafting, the cultured epidermis is maintained in the absence of dermal papillae and rete ridges, which only develop months later (Compton et al., 1998; Pellegrini et al., 1999). Thus the patterned array of keratinocytes may signal to locate dermal papillae, rather than dermal papillae defining the location of stem cells.

### Stem Cell Aging and the Origin of Meroclones

As discussed above, sub-cloning of colonies derived from single human keratinocytes can disclose the proliferative potential of the cell that founded the primary clone. Paraclones give rise to microscopic colonies with a broad distribution of sizes that are characteristic of the CP cell compartment, but perplexingly there are two types of macroscopic colony. Large circular holoclones have a higher proliferative potential when subcloned compared with irregularly shaped meroclones (Barrandon and Green, 1987b). This observation is significant as it offers a potential insight into human epidermal stem cell aging. In skin cultured from newborns, one third of clonogenic cells generate holoclones and 60% meroclones; the corresponding figures for epidermis from older adults are 0-3% holoclones and 10-30% meroclones (Barrandon and Green, 1987b). Aged human skin thus lacks the ability to produce holoclones and contains relatively few meroclones. Holoclone colonies have long been viewed as originating from stem cells, but the nature of the cells which generate the wrinkle-edged meroclone colonies has remained a puzzle.

To what extent does the model shed light on the behaviour of the cells that generate meroclones? Committed progenitor cells are incapable of generating colonies of the size of holoclone or meroclone colonies, indicating that these derive from stem cells. Strikingly, both the clone size and the morphology of the clone edge are sensitive indicators of stem cell behaviour. Meroclones may be smaller than holoclones for two reasons, a slower rate of cell division, and/or an increased rate of, stem cell differentiation. While a decreased rate of stem cell division will not affect the smooth edge morphology, an increased rate of differentiation will lead to the accumulation of CP cells at the clone margins, leading to the growth of clones with a wrinkled edge (see FIG. 12 and supplementary section S-VI). Applying the principles of the stem/CP cell model, we find that such an accumulation of CP and post-mitotic cells occurs when the rate of stem cell differentiation is increased by only a modest fraction: even if just 10% of stem cell divisions generate CP rather than stem cells, the predicted clone shape has a striking resemblance to that observed in meroclones (FIG. 12). Indeed, as expected for an increased rate of differentiation, the edges of wrinkled colonies are found to have a lower proportion of stem cells than the stem-cell rich holoclones (FIG. 12A-C). In addition to explaining an in vitro phenomenon, these results suggest that the loss of holoclones at the expense of meroclones in cultures of aged skin may reflect a decline in the stem cell potential for self-renewal.

### CONCLUSION

In conclusion, by drawing on a range of experimental data, we have elucidated the mechanisms of cell fate responsible for the maintenance of human interfollicular epidermis. In normal (uninjured) tissue, epidermis is largely maintained through the stochastic division and differentiation of a committed progenitor cell population. The combination of stem cell adhesion and density-regulated cell division facilitates the aggregation and quiescence of stem cell rich clusters, thereby protecting them from the risk of oncogenic mutation during DNA replication whilst allowing their mobilisation in response to injury. This pattern of regulation explains the potential of stem cells to reconstitute tissue in culture, and the relationship between colony shape and proliferative potential observed in sub-cloning experiments. As a signature of stem cell regulation and fate, pattern morphology provides a means to explore the action of drug treatments or genetic modification on these processes.

### Methods

### Keratinocyte Culture Classification

Colonies smaller than one quarter of the average size were defined as type I clones, while clones that were larger than or equal to the exponentially-growing average size were defined as type II. The remaining clones were classified according to Ki67 expression, with type I clones containing fewer than 50% cycling cells. A fit to the entire clone size distribution was then used to infer the proportion of differentiated two-cell clones belonging to the type I and type II sub-groups (see supplementary section S-I).

### Hydrodynamic Theory of Epidermal Maintenance

The processes of cell division and differentiation in the stem/CP cell model can be related to the rates, R_{S}=[γ^{(S)}_{SS}−γ^{(S)}_{A}]c_{S}, R_{A}=γ^{(S)}_{A}c_{S}−Δc_{A} and R_{B}=Γc_{A}−γ^{(B)}c_{B}, where S, A and B denote stem, CP and post-mitotic cells respectively. Here, γ^{(X)}_{YZ} is the average rate division rate of cell type X into two daughter cells (types Y, Z). γ^{(S)}_{A} represents the differentiation rate of stem cells into CP cells; Δ=γ^{(A)}_{BB}−γ^{(A)}_{AA} represents the effective differentiation rate of CP cells into post-mitotic cells, Γ=2 γ^{(A)}_{BB}+γ^{(A)}_{AB} denotes the net rate at which CP cells generate post-mitotic cells, and γ^{(B)} is the rate of migration of post-mitotic cells from the basal layer. During homeostasis, the cell division rates are regulated by the local cell density such that γ^{(X)}_{YZ}(r,t)=[1−c(r,t)] r^{(X)}_{YZ}, where c(r,t)=c_{S}(r,t)+c_{A}(r,t)+c_{B}(r,t) is the total local cell density expressed in units of the average cell area, and r^{(X)}_{YZ} denotes the bare division rate (supplementary section S-II).

Defining a local energy density f[{c_{X}}], the flux is obtained as,

$${J}_{X} = - σ {\sum }_{Y = S , A , B}^{ } {c}_{X} ( {δ}_{XY} - {c}_{Y} ) ∇ ( \frac{\partial f}{\partial {c}_{Y}} ) .$$

Here δ_{XY} denotes the Kroenecker delta symbol, and the sum over Y=S, A, and B ensures that the different cell types move in contrary directions so that the local cell density remains bound (c(r,t)<1). Both the diffusive cell motion and the effect of stem cell adhesion can be captured by a free energy density of Cahn-Hilliard type (Cahn and Hilliard, 1958),

$$f [ \{ {c}_{X} \} ] = {\sum }_{X} {c}_{X} ln {c}_{X} + ( 1 - c ) ln ( 1 - c ) - \frac{J}{2} [ {c}_{S}^{2} - {α ( ∇ {c}_{S} )}^{2} ] .$$

The first two terms give rise to a diffusive cell dynamics while the final term captures the effect of stem cell adhesion. The dimensionless parameter, J, characterises the strength of adhesion, and the constant Δ is a measure of the “surface tension” of stem cells, i.e. a measure of the smoothness of the boundaries of stem cell clusters. For simplicity, we have assumed that the different cell populations are characterised by the same mobility, σ. Numerical and analytical methods used to solve the hydrodynamic equations (1) are detailed in the supplementary section S-III.

In the steady state, the balance between cell division and differentiation leads to the following relations (supplementary section S-III): φ_{A}Γ=φ_{B}γ^{(B)}, and φ_{S}γ^{(S)}_{A}=φ_{A}Δ. From the latter, the ratio φ_{S}/φ_{A}=40/60 found from culture gives an estimate for the CP cell imbalance in terms of the stem cell differentiation rate, viz. Δ≈0.67 γ^{(S)}_{A}. For the numerical solution to Eq. (1) in FIG. 8, the following rates were used: γ^{(S)}_{A}=0.01, r^{(S)}_{SS}/r_{Γ}=0.02, r_{Δ}/r_{Γ}=0.01, and σ=4, where r_{Γ} and r_{Δ} are the bare division rates as they enter into Eq. (1), r_{Γ}=2r^{(A)}_{BB}+r^{(A)}_{AB} and r_{Δ}=r^{(A)}_{BB}-r^{(A)}_{AA}. The precise values of J, r_{Γ} and α are unimportant, provided that J and r_{Γ} are much greater than unity, and α˜1. We used the values J=12, J, r_{Γ}=200 and α=0.33. Here, all lengths are measured in units of the cell diameter, and all times are measured in units of the post-mitotic cell migration time, 1/γ^{(B)}.

### Further Predictions and Comparisons:

FIG. S9: Parameter dependence of the patterned steady-state morphology, demonstrated by plotting (a) A², α_{S}, and (b-d) α_{S}/A², c̅_{A}/ c̅_{B} against variations in the system parameters γ_{A}^{(S)} (a, b), r_{Δ} (c) and r_{SS}^{(S)} (d). The solid curves correspond to the analytic approximations given by Eqs. (S5), (S6) and (S7). The solid data points (red) were obtained from the numerical solution to the system equations (1), as described in the main text. Error bars indicate the tolerance of the system to variations in the pattern wavelength, as estimated by varying the initial conditions and then testing the stability of the patterned state. Referring to section S-V, the crosses (x) indicate the results obtained from cellular automata simulations of the stem/CP model that is capable of accounting for the effects of fluctuations. The plots correspond to the parameter sets described in the methods section of the main text (for the coarse-grained model) and in the caption of FIG. S12 (for the cellular automata). To evaluate the analytical solution, the values of W and Ω were estimated to be W=2.5, Ω=1.5.

FIG. S12: Examples of steady-state basal layer morphology obtained from cellular automata simulations of processes (S11)-(S15), showing the effects of increasing the stem cell differentiation rate (γ_{A}^{(S)}/γ^{(B)}=0.01 (a), 0.02 (b), 0.04 (c) and 0.08 (d)). Stem cells (green) form irregular domains within a background of committed progeniter (type A) cells (red) and post-mitotic (type B) cells (grey). The panels correspond to the data points (x) shown in FIG. S9(a, b). Parameters used for the simulation are the same as for FIG. 5 (see methods section in the main text), except for the following changes that take advantage of the faster simulation time but do not affect the predicted basal layer morphology: The vacancy diffusion rate is restored to the physical limit χ=100(>>1) and we set r_{AA}^{(A)}/r_{Γ}=0.1. The ‘bare’ division rate r_{Γ} is increased to r_{Γ}=6000, while the ratios of r_{SS}^{(S)}/r_{Γ}, r_{Δ}/r_{Γ} are unchanged. The latter changes have the effect of minimising the vacancy density (1−c) without otherwise altering the dynamics.

FIG. S13: Further examples of steady-state basal layer morphology obtained from cellular automata simulations, showing the effects of increasing the CP cell differentiation rate r_{Δ}/r_{Γ}, with r_{Δ}/r_{Γ}=0.01 (a), 0.02 (b), 0.04 (c) and 0.08 (d)). The panels correspond to the data points (x) shown in FIG. S9(c). The stem cell differentiation rate was held constant at γ_{A}^{(S)}/γ^{(B)}=0.04. See caption of FIG. S12 for legend and full parameter set.

FIG. S14: Further examples of steady-state basal layer morphology obtained from cellular automata simulations, showing the effects of increasing the bare stem cell division rate r_{SS}^{(S)}/r_{Γ}, with r_{SS}^{(S)}/r_{Γ}=0.01 (a), 0.02 (b), 0.04 (c) and 0.08 (d)). The panels correspond to the data points (x) shown in FIG. S9(d). The stem and CP cell differentiation rates were held constant at γ_{A}^{(S)}/γ^{(B)}=0.04 and r_{Δ}/r_{Γ}=0.02 respectively. See caption of FIG. S12 for legend and full parameter set.

### References to Example 8

Barrandon, Y., and Green, H. (1987a). Cell migration is essential for sustained growth of keratinocyte colonies: the roles of transforming growth factor-alpha and epidermal growth factor. Cell 50, 1131-1137.
Barrandon, Y., and Green, H. (1987b). Three clonal types of keratinocyte with different capacities for multiplication. Proc Natl Acad Sci USA 84, 2302-2306.
Braun, K. M., Niemann, C., Jensen, U. B., Sundberg, J. P., Silva-Vargas, V., and Watt, F. M. (2003). Manipulation of stem cell proliferation and lineage commitment: visualisation of label-retaining cells in wholemounts of mouse epidermis. Development 130, 5241-5255.
Cahn, J. W.; and Hilliard, J. E. (1958). Free Energy of a Nonuniform System. I. Interfacial Free Energy. The Journal of Chemical Physics 28, 258-267.
Claudinot, S., Nicolas, M., Oshima, H., Rochat, A., and Barrandon, Y. (2005). Long-term renewal of hair follicles from clonogenic multipotent stem cells. Proc Natl Acad Sci USA 102, 14677-14682.
Clayton, E., Doupe, D. P., Klein, A. M., Winton, D. J., Simons, B. D., and Jones, P. H. (2007). A single type of progenitor cell maintains normal epidermis. Nature 446, 185-189.
Compton, C. C., Nadire, K. B., Regauer, S., Simon, M., Warland, G., O'Connor, N. E., Gallico, G. G., and Landry, D. B. (1998). Cultured human sole-derived keratinocyte grafts re-express site-specific differentiation after transplantation. Differentiation 64, 45-53.
Elliott, C. M., and Garcke, H. (1997). Diffusional phase transitions in multicomponent systems with a concentration dependent mobility matrix. Physica D 109, 242-256.
Estrach, S., Legg, J., and Watt, F. M. (2007). Syntenin mediates Delta1-induced cohesiveness of epidermal stem cells in culture. J Cell Sci 120, 2944-2952.
Fuchs, E. (2007). Scratching the surface of skin development. Nature 445, 834-842.
Ghazizadeh, S., and Taichman, L. B. (2005). Organization of stem cells and their progeny in human epidermis. J Invest Dermatol 124, 367-372.
Giacomin, G., and Lebowitz, J. L. (1996). Exact Macroscopic Description of Phase Segregation in Model Alloys with Long Range Interactions. Phys Rev Lett 76, 1094-1097.
Hall, P. A., and Watt, F. M. (1989). Stem cells: the generation and maintenance of cellular diversity. Development 106, 619-633.
Ito, M., Liu, Y., Yang, Z., Nguyen, J., Liang, F., Morris, R. J., and Cotsarelis, G. (2005). Stem cells in the hair follicle bulge contribute to wound repair but not to homeostasis of the epidermis. Nat Med 11, 1351-1354.
Ito, M., Yang, Z., Andl, T., Cui, C., Kim, N., Millar, S. E., and Cotsarelis, G. (2007). Wnt-dependent de novo hair follicle regeneration in adult mouse skin after wounding. Nature 447, 316-320.
Jensen, K. B., and Watt, F. M. (2006). Single-cell expression profiling of human epidermal stem and transit-amplifying cells: Lrig1 is a regulator of stem cell quiescence. Proc Natl Acad Sci USA 103, 11958-11963.
Jensen, U. B., Lowell, S., and Watt, F. M. (1999). The spatial relationship between stem cells and their progeny in the basal layer of human epidermis: a new view based on whole-mount labelling and lineage analysis. Development 126, 2409-2418.
Jones, P. H., Harper, S., and Watt, F. M. (1995). Stem cell patterning and fate in human epidermis. Cell 80, 83-93.
Jones, P. H., Simons, B. D., and Watt, F. M. (2007): Sic Transit Gloria: Farewell to the Epidermal Transit Amplifying Cell? Cell Stem Cell 1, 371-381.
Jones, P. H., and Watt, F. M. (1993). Separation of human epidermal stem cells from transit amplifying cells on the basis of differences in integrin function and expression. Cell 73, 713-724.
Klein, A. M., Doupe, D. P., Jones, P. H., and Simons, B. D. (2007). Kinetics of cell division in epidermal maintenance. Phys Rev E 76, 021910.
Koster, M. I., and Roop, D. R. (2005). Asymmetric cell division in skin development: a new look at an old observation. Dev Cell 9, 444-446.
Lajtha, L. G. (1979). Stem cell concepts. Differentiation 14, 23-34.
Legg, J., Jensen, U. B., Broad, S., Leigh, I., and Watt, F. M. (2003). Role of melanoma chondroitin sulphate proteoglycan in patterning stem cells in human interfollicular epidermis. Development 130, 6049-6063.
Levy, V., Lindon, C., Harfe, B. D., and Morgan, B. A. (2005). Distinct stem cell populations regenerate the follicle and interfollicular epidermis. Dev Cell 9, 855-861.
Lowell, S., Jones, P., Le Roux, I., Dunne; J., and Watt, F. M. (2000). Stimulation of human epidermal differentiation by delta-notch signalling at the boundaries of stem-cell clusters. Curr Biol 10, 491-500.
Lowell, S., and Watt, F. M. (2001). Delta regulates keratinocyte spreading and motility independently of differentiation. Mech Dev 107, 133-140.
Mackenzie, I. C. (1970). Relationship between mitosis and the ordered structure of the stratum corneum in mouse epidermis. Nature 226, 653-655.
Morris, R. J., Liu, Y., Marles, L., Yang, Z., Trempus, C., Li, S., Lin, J. S., Sawicki, J. A., and Cotsarelis, G. (2004). Capturing and profiling adult hair follicle stem cells. Nat Biotechnol 22, 411-417.
Morrison, S. J., and Spradling, A. C. (2008). Stem cells and niches: mechanisms that promote stem cell maintenance throughout life. Cell 132, 598-611.
Pellegrini, G., Ranno, R., Stracuzzi, G., Bondanza, S., Guerra, L., Zambruno, G., Micali, G., and De Luca, M. (1999). The control of epidermal stem cells (holoclones) in the treatment of massive full-thickness burns with autologous keratinocytes cultured on fibrin. Transplantation 68, 868-879.
Potten, C. S. (1974). The epidermal proliferative unit: the possible role of the central basal cell. Cell Tissue Kinet 7, 77-88.
Potten, C. S. (1981). Cell replacement in epidermis (keratopoiesis) via discrete units of proliferation. Int Rev Cytol 69, 271-318.
Rheinwald, J. G., and Green, H. (1975). Serial cultivation of strains of human epidermal keratinocytes: the formation of keratinizing colonies from single cells. Cell 6, 331-343.
Savill, N. J., and Sherratt, J. A. (2003). Control of epidermal stem cell clusters by Notch-mediated lateral induction. Dev Biol 258, 141-153.
Sherwood, R. I., Christensen, J. L., Conboy, I. M., Conboy, M. J., Rando, T. A., Weissman, I. L., and Wagers, A. J. (2004). Isolation of adult mouse myogenic progenitors: functional heterogeneity of cells within and engrafting skeletal muscle. Cell 119, 543-554.
Shraiman, B. I. (2005). Mechanical feedback as a possible regulator of tissue growth. Proc Natl Acad Sci USA 102, 3318-3323.
Smart, I. H. (1970). Variation in the plane of cell cleavage during the process of stratification in the mouse epidermis. Br J Dermatol 82, 276-282.
Tumbar, T., Guasch, G., Greco, V., Blanpain, C., Lowly, W. E., Rendl, M., and Fuchs, E. (2004). Defining the epithelial stem cell niche in skin. Science 303, 359-363.
Wan, H., Stone, M. G., Simpson, C., Reynolds, L. E., Marshall, J. F., Hart, I. R., Hodivala-Dilke, K. M., and Eady, R. A. (2003). Desmosomal proteins, including desmoglein 3, serve as novel negative markers for epidermal stem cell-containing population of keratinocytes. J Cell Sci 116, 4239-4248.

### References to Examples 1 to 7

1. Stark, H. J. et al. Epidermal homeostasis in long-term scaffold-enforced skin equivalents. J Invest Dermatol 126 Suppl, 93-105 (2006).
2. Parenteau, N. L. et al. Epidermis generated in vitro: practical considerations and applications. J Cell Biochem 45, 245-51 (1991).
3. Stark, H. J. et al. Authentic fibroblast matrix in dermal equivalents normalises epidermal histogenesis and dermoepidermal junction in organotypic co-culture. Eur J Cell Biol 83, 631-45 (2004).
4. Boehnke, K. et al. Effects of fibroblasts and microenvironment on epidermal regeneration and tissue function in long-term skin equivalents. Eur J Cell Biol (2007).
5. Bell, E. et al. Development and use of a living skin equivalent. Plast Reconstr Surg 67, 386-92 (1981).
6. Serrano, F. et al. A comparison of targeting performance of oncoretroviral versus lentiviral vectors on human keratinocytes. Hum Gene Ther 14, 1579-85 (2003).
7. Mildner, M. et al. Gene silencing in a human organotypic skin model. Biochem Biophys Res Commun 348, 76-82 (2006).
8. Garlick, J. A. & Taichman, L. B. Fate of human keratinocytes during reepithelialization, in an organotypic culture model. Lab Invest 70, 916-24 (1994).
9. Legg, J., Jensen, U. B., Broad, S., Leigh, I. & Watt, F. M. Role of melanoma chondroitin sulphate proteoglycan in patterning stem cells in human interfollicular epidermis. Development 130, 6049-63 (2003).
10. Jones, P. H. & Watt, F. M. Separation of human epidermal stem cells from transit amplifying cells on the basis of differences in integrin function and expression. Cell 73, 713-24 (1993).
11. Jones, P. H., Harper, S. & Watt, F. M. Stem cell patterning and fate in human epidermis. Cell 80, 83-93 (1995).
12. Jensen, U. B., Lowell, S. & Watt, F. M. The spatial relationship between stem cells and their progeny in the basal layer of human epidermis: a new view based on whole-mount labelling and lineage analysis. Development 126, 2409-18 (1999).
13. Jensen, K. B. & Watt, F. M. Single-cell expression profiling of human epidermal stem and transit-amplifying cells: Lrig1 is a regulator of stem cell quiescence. Proc Natl Acad Sci USA 103, 11958-63 (2006).
14. Williams, G. H. et al. Improved cervical smear assessment using antibodies against proteins that regulate DNA replication. Proc Natl Acad Sci USA 95, 14932-7 (1998).
15. Madine, M. A. et al. The roles of the MCM, ORC, and Cdc6 proteins in determining the replication competence of chromatin in quiescent cells. J Struct Biol 129, 198-210 (2000).
16. van den Bogaerdt, A. J. et al. Upside-down transfer of porcine keratinocytes from a porous, synthetic dressing to experimental full-thickness wounds. Wound Repair Regen 12, 225-34 (2004).
17. Ghazizadeh, S. & Taichman, L. B. Organization of stem cells and their progeny in human epidermis. J Invest Dermatol 124, 367-72 (2005).
18. Lowell, S., Jones, P., Le Roux, I., Dunne, J. & Watt, F. M. Stimulation of human epidermal differentiation by delta-notch signalling at the boundaries of stem-cell clusters. Curr Biol 10, 491-500 (2000).
19. Tiscornia, G., Tergaonkar, V., Galimi, F. & Verma, I. M. CRE recombinase-inducible RNA interference mediated by lentiviral vectors. Proc Natl Acad Sci USA 101, 7347-51 (2004).
20. Hanakawa, Y, et al. Cre-loxP adenovirus-mediated foreign gene expression in skin-equivalent keratinocytes. Br J Dermatol 152, 1391-2 (2005).
21. Clayton, E. et al. A single type of progenitor cell maintains normal epidermis. Nature 446, 185-189 (2007).
22. Barrandon, Y. & Green, H. Three clonal types of keratinocyte with different capacities for multiplication. Proc Natl Acad Sci USA 84, 2302-6 (1987).

### REFERENCES

Barrandon, Y. and Green, H. (1987). Three clonal types of keratinocyte with different capacities for multiplication. Proc Natl Acad Sci USA 84, 2302-6.
Bickenbach, J. R. (1981). Identification and behavior of label-retaining cells in oral mucosa and skin. J Dent Res 60 Spec No C, 1611-20.
Bickenbach, J. R. and Chism, E. (1998). Selection and extended growth of murine epidermal stem cells in culture. Exp Cell Res 244, 184-95.
Braun, K. M., Niemann, C., Jensen, U. B., Sundberg, J. P., Silva-Vargas, V. and Watt, F. M. (2003). Manipulation of stem cell proliferation and lineage commitment: visualisation of label-retaining cells in wholemounts of mouse epidermis. Development 130, 5241-55.
Clayton, E., Doupe, D. P., Klein, A. M., Winton, D. J., Simons, B. D. and Jones, P. H. (2007). A single type of progenitor cell maintains normal epidermis. Nature 446, 185-189.
Fuchs, E. (2007). Scratching the surface of skin development. Nature 445, 834-42.
Ito, M., Liu, Y., Yang, Z., Nguyen, J., Liang, F., Morris, R. J. and Cotsarelis, G. (2005). Stem cells in the hair follicle bulge contribute to wound repair but not to homeostasis of the epidermis. Nat Med 11, 1351-4.
Jensen, K. B. and Watt, F. M. (2006). Single-cell expression profiling of human epidermal stem and transit-amplifying cells: Lrig1 is a regulator of stem cell quiescence. Proc Natl Acad Sci USA 103, 11958-63.
Jensen, U. B., Lowell, S. and Watt, F. M. (1999). The spatial relationship between stem cells and their progeny in the basal layer of human epidermis: a new view based on whole-mount labelling and lineage analysis. Development 126, 2409-18.
Jones, J. E. and Corwin, J. T. (1993). Replacement of lateral line sensory organs during tail regeneration in salamanders: identification of progenitor cells and analysis of leukocyte activity. J Neurosci 13, 1022-34.
Jones, P. H., Harper, S. and Watt, F. M. (1995). Stem cell patterning and fate in human epidermis. Cell 80, 83-93.
Jones, P. H. and Watt, F. M. (1993). Separation of human epidermal stem cells from transit amplifying cells on the basis of differences in integrin function and expression. Cell 73, 713-24.
Klein, A. M., Doupe, D. P., Jones, P. H. and Simons, B. D. (In press). Kinetics of cell division in epidermal maintenance. Physical Review E.
Legg, J., Jensen, U. B., Broad, S., Leigh, I. and Watt, F. M. (2003). Role of melanoma chondroitin sulphate proteoglycan in patterning stem cells in human interfollicular epidermis. Development 130, 6049-63.
Levy, V., Lindon, C., Harfe, B. D. and Morgan, B. A. (2005). Distinct stem cell populations regenerate the follicle and interfollicular epidermis. Dev Cell 9, 855-61.
Lowell, S., Jones, P., Le Roux, I., Dunne, J. and Watt, F. M. (2000). Stimulation of human epidermal differentiation by delta-notch signalling at the boundaries of stem-cell clusters. Curr Biol 10, 491-500.
Lowell, S. and Watt, F. M. (2001). Delta regulates keratinocyte spreading and motility independently of differentiation. Mech Dev 107, 133-40.
Morris, R. J., Liu, Y., Marks, L., Yang, Z., Trempus, C., Li, S., Lin, J. S., Sawicki, J. A. and Cotsarelis, G. (2004). Capturing and profiling adult hair follicle stem cells. Nat Biotechnol 22, 411-7.
Potten, C. S. (1981). Cell replacement in epidermis (keratopoiesis) via discrete units of proliferation. Int Rev Cytol 69, 271-318.
Savill, N. J. (2003). Mathematical models of hierarchically structured cell populations under equilibrium with application to the epidermis. Cell Prolif 36, 1-26.
Savill, N. J. and Sherratt, J. A. (2003). Control of epidermal stem cell clusters by Notch-mediated lateral induction. Dev Biol 258, 141-53.
Tumbar, T., Guasch, G., Greco, V., Blanpain, C., Lowry, W. E., Rendl; M. and Fuchs, E. (2004). Defining the epithelial stem cell niche in skin. Science 303, 359-63.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method of detecting an altered behaviour in a population of cells, said method comprising determining at least one of the following characteristics of the population of cells;
(i) the proportion of stem cells, proliferating cells and differentiated cells in said cell population; or
(ii) the size of stem cell clusters in said cell population; or
(iii) the separation of stem cell clusters in said cell population; and
comparing said at least one characteristic to a reference value,
wherein a difference between the determined value and the reference value indicates an altered behaviour in said population of cells.

2. A method according to claim 1 wherein each of the three characteristics (i) to (iii) are determined, and wherein each of said three characteristics is determined by measurement.

3. A method according to claim 1 wherein said behaviour is selected from the group consisting of stem cell division rate, stem cell differentiation rate, stem cell adhesion capacity, committed progenitor cell division rate and differentiated cell migration rate.

4. A method according to claim 1 wherein when the characteristic determined is the size of stem cell clusters in the population, it is determined as the average diameter of stem cell clusters in millimetres.

5. A method according to claim 1 wherein when the characteristic determined is the separation of stem cell clusters in the population, it is determined as the average distance between the outer edges of discrete adjacent stem cell clusters in microns.

6. A method according to claim 1 wherein said population of cells is a population of mammalian epidermal cells.

7. A method according to claim 1 wherein said population of cells is a population of basal layer cells.

8. A method according to claim 1 wherein said population of cells comprises an organotypic keratinocyte culture.

9. A method according to claim 1 wherein said cells are human cells.

10. A method according to claim 1 wherein said reference value is generated from a control population of cells.

11. A method according to claim 1 wherein said reference value is predicted or described by the equation
$$\frac{d {c}_{x}}{d t} = - ∇ \cdot {J}_{x} + {R}_{x} , \\ where {J}_{X} = - {\sum }_{Y = S , A , B , Φ}^{ } {M}_{XY} ∇ ( \frac{δ F}{δ {c}_{Y}} ) . \qquad ( 1 )$$

12. A method for assessing the effect of a treatment on behaviour in a population of cells, said method comprising
(i) providing a first and a second population of cells;
(ii) applying the treatment to said first population of cells;
(iii) incubating said first and second populations of cells;
(iv) detecting an altered behaviour in said first population of cells according to claim,
wherein said reference value is the value determined for said second population of cells, and wherein detection of altered behaviour in said first population of cells indicates that said treatment has an effect on behaviour in said population of cells.

13. A method according to claim 1 further comprising performing a clonal analysis.

14. A method according to claim 13 wherein said clonal analysis comprises determining the clone size distribution of said population of cells, and comparing the clone size distribution measured to a reference clone size distribution at a corresponding time point t predicted or described by the equation;
$$\frac{d {P}_{{n}_{A} , {n}_{B}}}{d t} = λ \{ r [ ( {n}_{A} - 1 ) {P}_{{n}_{A} - 1 , {n}_{B}} + ( {n}_{A} + 1 ) {P}_{{n}_{A} + 1 , {n}_{B} - 2} ] + ( 1 - 2 r ) {n}_{A} {P}_{{n}_{A} , {n}_{B} - 1} - {n}_{A} {P}_{{n}_{A} , {n}_{B}} \} + r [ ( {n}_{B} + 1 ) {P}_{{n}_{A} , {n}_{B} + 1} - {n}_{B} {P}_{{n}_{A} , {n}_{B}} ]$$
wherein a difference between the measured clone size distribution and the predicted or described clone size distribution further indicates an altered proliferation or differentiation behaviour of said cells.

15. (canceled)
